(19) United States
(12) Patent Application Publication
(10) Pub. No.: US 2013/0053434 A1
(43) Pub. Date: Feb. 28, 2013

(54) IRREVERSIBLE INHIBITORS USEFUL FOR THE TREATMENT OF KINASE-RELATED PATHOLOGIES

(71) Applicants: Nicolas Winssinger, Strasbourg (FR); Sofia Barluenga, Strasbourg (FR)

(72) Inventors: Nicolas Winssinger, Strasbourg (FR); Sofia Barluenga, Strasbourg (FR)

(73) Assignees: CENTRE NATIONAL DE LA RECHERCHE SCIENTIFIQUE, Paris (FR); UNIVERSITE DE STRASBOURG, Strasbourg (FR)

(21) Appl. No.: 13/395,532

(22) PCT Filed: Sep. 28, 2010
(86) PCT No.: PCT/EP2010/064305

§ 371 Date: Nov. 2, 2012

Related U.S. Application Data
(60) Provisional application No. 61/246,361, filed on Sep. 28, 2009.
(60) Provisional application No. 61/306,669, filed on Feb. 22, 2010.

(51) Int. Cl. A61K 31/365; A61P 35/04; A61P 35/00; C07D 313/00; C07D 321/00

(52) U.S. Cl. 514/450; 549/270; 549/267

(57) ABSTRACT

The present invention provides new compounds having a kinase inhibitory activity and useful for treating cancer.

### FIELD OF THE INVENTION

The present invention relates to new compounds useful as drug, in particular in the oncology field.

### BACKGROUND OF THE INVENTION

Phosphorylation of proteins is one of the most prevalent cellular mechanisms for regulating protein function in a rapid and reversible fashion. There are approximately 518 protein kinases encoded in the human genome as well as a smaller set of protein phosphatases which can work synergistically or concurrently at various levels in cellular pathways, thus resulting in a tremendous combinatorial output. Virtually every signal transduction pathway implicates kinases and as much as 30% of all human proteins may be modified by them. A number of pathologies ranging from oncology to inflammation and neurodegenerative diseases can be attributed to a dysfunctional kinase. Accordingly, kinases have become one of the most intensively pursued classes of proteins for drug discovery, the vast majority being currently investigated for the treatment of cancer. To date, 11 kinase inhibitors have received FDA approval and there are approximately 30 distinct kinase targets being developed at the level of phase I clinical trials.

Four natural resorcylic acid lactones (RAL) containing a cis-enone moiety have been reported to be potent and irreversible kinase inhibitors (N. Winssinger, S. Barluenga, Chem. Commun. 2007, 22-36; S. Barluenga, et al, C. R. Chem. 2008, 11, 1306-1317; T. Hofmann, K.-H. Altmann, C. R. Chem. 2008, 11, 1318-1335.) (L-783277 also named FR265082; LL-Z1640-2 also named 5Z-7-oxozeaenol and FR148083; radicicol A and hypothemycin). Two of these compounds (5Z-7-oxozeaenol and hypothemycin) have already been shown to be effective in animal models (J. Ninomiya-Tsuji, et al, J. Biol. Chem. 2003, 278, 18485-18490.; H. Tanaka, et al, Jap. J. Cancer Res. 1999, 90, 1139-1145.). As such, these cis-enone RAL represent a pharmacologically validated starting point for kinase inhibition. More recently, it has been shown by Santi and coworkers that hypothemycin irreversibly inactivates ERK2 by forming a covalent Michael adduct with the cys166 positioned in the ATP-binding pocket of this kinase (A. Schirmer, et al, Proc. Natl. Acad. Sci. USA 2006, 103, 4234-4239). A structure-bioinformatics analysis of the kinome revealed that 46 out of the 518 putative kinases contained a cysteine residue adequately positioned to participate in the Michael addition onto the cis-enone of hypothemycin. Nevertheless, it was clearly shown that a significant difference in the kinetics of inactivation existed amongst 16 out of the 46 tested kinases. Our profile of the related chloro-radicicol A against 127 kinases showed that this compound inhibited exclusively kinases from this subset of 46, however not all were inhibited (GSK3β for example was not inhibited) (P. Y. Dakas, et al, Angew. Chem. Int. Ed. Engl. 2007, 46, 6899-6902). Furthermore it was shown by Nakajima and Miyake that there was a significant difference in activity (50 fold) between LL-Z1640-2 (FR148083) and L-783277 (FR265082) in their inhibition of ERK2 suggesting that a good level of selectivity may be achieved within this subset of the kinome as L-783277 was reported to be a potent MEK inhibitor (4 nM) compared to LL-Z1640-2 (411 nM).

Analogues of radicicol A have been disclosed in WO 03/076424, WO 06/036941 and WO 08/149,244.

However, there is still a strong need for kinase inhibitors that have improved potency and selectivity.

### SUMMARY OF THE INVENTION

The present invention provides new compounds with improved potency and selectivity as kinase inhibitors. In particular, they predominantly inhibit the vascular endothelial growth factor receptors (VEGFRs) and platelet-derived growth factor receptors (PDGFRs). Accordingly, they are useful as drug, in particular of therapeutic interest for treating cancer. A particular group of compounds disclosed herein present an advantage in comparison with recent approved small molecule inhibitors (sorafenib and sunitinib) targeting the VEGF receptors. While sunitinib did promote lung metastasis in an in vivo model, compounds of Formula II strongly inhibited lung metastasis. Therefore, compounds of Formula II are of a great therapeutic interest.

The present invention relates to a compound of formula I, a tautomer or pharmaceutically acceptable salt, solvate, or ester thereof,

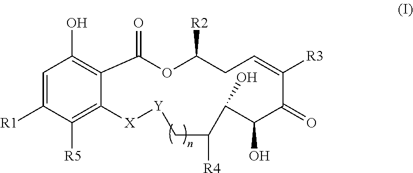

n is 0 or 1;

R1 is selected from the group consisting of hydrogen, halogen, cyano, —OR, —NRR′, —NRS(O)R′, —NRS(O₂)R′, —SR, —S(O)R, —S(O₂)R, —OC(O)R, —C(O)R, —C(O)OR, —NRC(O)R′, —C(O)NRR′, —OC(O)OR, aliphatic, heteroaliphatic, acyl, aryl, heteroaryl, alkylaryl, arylalkyl, alkylheteroaryl and heteroarylalkyl;

R2 is hydrogen, or a C₁-C₅ alkyl;

R3 is hydrogen or halogen;

R4 is hydrogen or hydroxyl;

R5 is hydrogen, halogen, OR, NHR, NH—COR,

R and R′, independently, are hydrogen, or a C₁-C₅ alkyl;

X—Y is CH═CH, CH₂—CH₂, O—CH₂, CH₂—O, S—CH₂, or CH₂—S, and,

provided that the compound is not a compound of Formula I with

n is 0, R1 is hydroxyl, R3, R4 and R5 are hydrogen, R2 is methyl, X—Y is O—CH₂ or CH₂—S; or

n is 0, R1 is hydroxyl, R4 and R5 are hydrogen, R2 is methyl, R3 is fluorine, X—Y is

O—CH₂; or

n is 0, R1 is methoxy, R2 is methyl, R3 is fluorine, R4 and R5 are hydrogen, X—Y is CH₂—S.

In a preferred embodiment, R1 is selected from the group consisting of —OR, —NHR and —SR.

In a particularly preferred embodiment, the invention relates to the compound having the formula II,

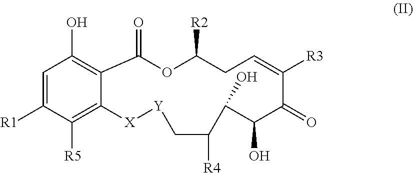

R1, R2, R3, R4, R5 and X—Y being as defined in compounds of Formula (I).

Preferably, the compound of Formula II has one or several of the following features:
R1 is —OR and R is selected from hydrogen, methyl, ethyl and isopropyl, more preferably R1 is hydroxyl or methoxy; and/or
R2 is selected from hydrogen, methyl, ethyl and isopropyl, more preferably from hydrogen or methyl, still more preferably is a methyl; and/or
R3 is hydrogen or fluorine, preferably hydrogen; and/or
R4 is hydrogen or hydroxyl, preferably hydrogen; and/or
R5 is hydrogen; and/or
X—Y is selected from CH═CH, CH₂—CH₂, O—CH₂, CH₂—O, S—CH, and CH—S, preferably from CH═CH, CH₂—CH₂, and O—CH₂; and/or

provided that the compound does not have concomitantly a hydrogen in R2 and O—CH₂ in X—Y.

More preferably, the compound of Formula II has one or several of the following features:
R1 is hydroxyl or methoxy; and/or
R2 is hydrogen or methyl; and/or
R3 is hydrogen; and/or
R4 is hydrogen; and/or
R5 is hydrogen; and/or
X—Y is selected from CH═CH, CH₂—CH₂, and O—CH₂;

provided that the compound does not have concomitantly a hydrogen in R2 and O—CH₂ in X—Y.

Still more preferably, in the compound of Formula II, X—Y is CH₂—CH₂.

In a particular embodiment of the compound of Formula II, R1 is hydroxyl or methoxy, R2, R3, R4 and R5 are hydrogen, and X—Y is CH═CH or CH₂—CH₂.

In another particular embodiment of the compound of Formula II, R1 is hydroxyl or methoxy, R2 is methyl, R3, R4 and R5 are hydrogen, and X—Y is selected from CH═CH, CH₂—CH₂, and O—CH₂.

Accordingly, the compound of Formula II may be selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and,
a compound of formula II, wherein R2, R3, R4 and R5 are hydrogen, R1 is methoxy and X—Y is CH₂—CH₂.

In another particularly preferred embodiment, the invention relates to the compound having the formula III,

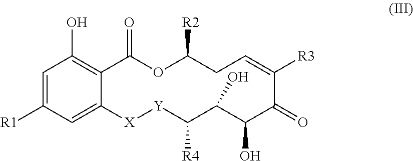

R1, R2, R3, R4 and X—Y being as defined in Formula (I).

Preferably, the compound of Formula (III) has one or several of the following features:
R1 is —OR and R is selected from hydrogen, methyl, ethyl and isopropyl, more preferably R1 is hydroxyl or methoxy; and/or
R2 is selected from hydrogen, methyl, ethyl and isopropyl, more preferably from hydrogen or methyl, still more preferably is a methyl; and/or
R3 is hydrogen or fluorine, preferably hydrogen; and/or
R4 is hydrogen or hydroxyl; and/or
X—Y is selected from CH═CH, CH₂—CH₂, O—CH₂, CH₂—O, S—CH, and CH—S, preferably from CH═CH, CH₂—CH₂, and O—CH₂.

More preferably, the compound of Formula (III) has one or several of the following features:
R1 is hydroxyl; and/or
R2 is hydrogen or methyl, preferably methyl; and/or
R3 is hydrogen or fluorine, preferably hydrogen; and/or
R4 is hydrogen or hydroxyl; and/or
X—Y is selected from CH═CH, CH₂—CH₂, and O—CH₂.

Still more preferably, in the compound of Formula III, R2 is methyl, ethyl and isopropyl, preferably methyl, and R4 is hydroxyl.

Accordingly, the compound of Formula III may be selected from the group consisting of:
a compound of formula III, wherein R1 is hydroxyl, R3 and R4 are hydrogen, R2 is methyl, and X—Y is CH₂—CH₂;
a compound of formula III, wherein R1 is hydroxyl, R3 is hydrogen, R2 is methyl, R4 is hydroxyl, and X—Y is O—CH₂;
a compound of formula III, wherein R1 is hydroxyl, R2, R3 and R4 are hydrogen, and X—Y is O—CH₂;
a compound of formula III, wherein R1 is hydroxyl, R2 and R3 are hydrogen, R4 is hydroxyl, and X—Y is O—CH₂; and,
a compound of formula III, wherein R1 is hydroxyl, R4 is hydrogen, R2 is methyl, R3 is fluorine, and X—Y is CH═CH.

The present also relates to any of the above-disclosed compounds as a drug. It further relates to a pharmaceutical composition comprising any of the above-disclosed compounds and a pharmaceutically acceptable carrier.

The present invention relates to any of the above-disclosed compounds for use in the treatment of cancer, to the use of any of the above-disclosed compounds for the preparation of a medicament for treating cancer, and to a method for treating cancer in a subject, comprising administering a therapeutic effective amount of any of the above-disclosed compounds to said subject.

In a more particular aspect, the present invention relates to any of the above-disclosed compounds of Formula (II) for use in the treatment of cancer while preventing metastasis occurrence, to the use of any of the above-disclosed compounds for the preparation of a medicament for treating cancer while preventing metastasis occurrence, and to a method for treating cancer while preventing metastasis occurrence in a subject, comprising administering a therapeutic effective amount of any of the above-disclosed compounds to said subject.

### BRIEF DESCRIPTION OF THE DRAWINGS

FIG. 1. Structure of natural resorcylic acid lactones bearing a cis-enone.

FIG. 2. General structure and synthetic planning of a library of cis-enone RAL.

FIG. 3. Synthesis of R_{F} tagged fragments 6a-d. a) PMBOC═NHCCl₃ (1.0 equiv), CSA (0.14 equiv), CH₂Cl₂, 23° C., 12 h, 72-86%; b) DIBAL-H (1.1 equiv), PhMe, −78° C., 1 h, 45-52%; c) CBr₄ (4.0 equiv), PPh₃ (8.0 equiv), CH₂Cl₂, 0° C., 45 min, 63%; d) nBuLi (2.0 equiv), THF, −78° C., 1 h, and 23° C., 1.5 h, 85%; e) HC₂MgBr (1.5 equiv), THF, −78° C. to 23° C., 12 h, 88%; f) EOM-Cl (3.0 equiv), iPr₂NEt (3.0 equiv), n-Bu₄N⁺I (cat), CH₂Cl₂, 23° C., 12 h, 89%; g) Cp₂ZrCl₂ (0.25 equiv), AlMe₃ (3.0 equiv), CH₂Cl₂, 23° C., 19 h and reflux 5 days; h) I₂ (1.5 equiv), THF, −30° C., 15 min, 62%. CSA=camphorsulfonic acid, Cp=cyclopentadienyl, DIBAL-H=diisobutylaluminium hydride, EOM=ethoxymethyl, PMB=p-methoxybenzyl, THF=tetrahydrofuran.

FIG. 4. Synthesis of fragments 7. a) 2-methoxypropene (2.0 equiv), pTsA (0.04 equiv), CaSO₄ (0.25 equiv), DMF, 0° C., 3 h, 60% or 2,2-dimethoxypropane (3.5 equiv), pTsA (0.02 equiv), acetone, 23° C., 12 h, 90%; b) LiAlH₄ (1.4 equiv), THF, 0° C. to 23° C., 2 h, 95%; c) TBDPS-Cl (0.9 equiv), imidazole (1.5 equiv), DMF, 23° C., 2-12 h, 66-99%; d) SO₃.Py complex (3.5 equiv), Et₃N (4.9 equiv), CH₂Cl₂/DMSO 4/1, 0° C. to 23° C., 0.5-1 h, 91-94%; e) BrPPh₃CH₃ (3.0 equiv), NaHDMS (2.8 equiv), THF, −78° C. to 23° C., 1-12 h, 72-86%; f) PivCl (2.0 equiv), Et₃N (4.0 equiv), DMAP (0.2 equiv), CH₂Cl₂, 0° C. to 23° C., 12 h, 93%; g) 9-BBN (2.2 equiv), THF, 0° C. to 23° C., 3.5 h, then 3N NaOH/H₂O₂, 0° C. to 23° C., 1.5 h, 94%; h) NaOMe (3.0 equiv), MeOH, 23° C., 16 h, 83%; i) BnOH (7.2 equiv), 23° C., 10 h, 96%; j) EOM-Cl (8.0 equiv), iPr₂NEt (8.0 equiv), nBu₄NI (cat), CH₂Cl₂, 23° C., 12 h, 95%; k) H₂, Pd/C (5 mol %), MeOH, 23° C., 5 h, 90%; 1) O₃, PPh₃ (2.0 equiv), CH₂Cl₂, −78° C., 1 h, 90%. Bn=benzyl, 9-BBN=9-borabicylo[3.3.1]nonane, DMAP=4-dimethylaminopyridine, DMF=N,N-dimethylformamide, DMSO=dimethylsulfoxide, EOM=ethoxymethyl, NaHDMS=sodium bis(trimethylsilyl)amide, Piv=pivaloyl, pTsA=p-toluene sulfonic acid, py=pyridine, TBAI=tetrabutylammonium iodide, TBDPS=tert-butyldiphenylsilyl, THF=tetrahydrofuran.

FIG. 5. Synthesis of compounds 5 (12 permutations) via fluorous mixture synthesis using three pools. a) nBuLi (1.1 equiv), THF, −78° C., 10 min, then 7 (1.2 equiv), −78° C., 30 to 60 min, 80%; b) BzCl (2.5 equiv), pyridine (2.5 equiv), CH₂Cl₂, 0° C. to 23° C., 4 h, 90%; c) TBAF (1.5 equiv), THF, 23° C., 3 h, 86-94%; d) H₂, Pd/CaCO₃ (1.72 equiv), MeOH, 23° C., 45 min, 90-96%; e) PPh₃ (1.5 equiv), imidazole (2.5 equiv), I₂ (1.5 equiv), THF, 0° C. ° C., 30 min, 70-93%; f) 6d (1.0 equiv), tBuLi (2.0 equiv), Et₂O, −78° C., 20 min, then 7 (1.2 equiv), pentane, −78° C. to 0° C., 7 h, >80%. Bz=benzoyl, TBAF=tetrabutylammonium fluoride, THF=tetrahydro furan.

FIG. 6. Synthesis of fragments 4. a) TFA (20.0 equiv), TFAA (7.6 equiv), acetone, 23° C., 12 h, 54%; b) EOM-Cl (4.0 equiv), iPr₂NEt (4.0 equiv), n-Bu₄N⁺I (cat), CH₂Cl₂, 23° C., 12 h, quant.; c) TMSCH₂CH₂OH (4.0 equiv), NaHDMS (4.4 equiv), THF, 0° C. to 23° C., 12 h, 69%; d) LDA (2.0 equiv), THF, −78° C., 30 min, (PhSe)₂ (0.9 equiv), THF, −78° C., 2 h, 75%. EOM=ethoxymethyl, LDA=lithium diisopropylamide, NaHDMS=sodium bis(trimethylsilyl)amide, TFA=trifluoro acetic acid, TFAA=trifluoro acetic anhydride, THF=tetrahydrofuran, TMSE=2-trimethylsilylethyl.

FIG. 7. Library synthesis. a) LDA (2.0 equiv), THF, −78° C., 10 min, then 5 (0.9 equiv), THF, −78° C., 2.0 h, 60%; b) LDA (2.0 equiv), THF/HMPA (10/1), −78° C., 10 min, then 5 (0.9 equiv), THF, −78° C., 30 min and then H₂O₂ (2.0 equiv), THF, 23° C., 2 h, 64-85%; c) K₂CO₃ (2.0 equiv), 5 (0.9 equiv), DMF, 100° C., 12 h, 94-100%; d) DDQ (1.2 equiv), CH₂Cl₂/H₂O (2/1), 23° C., 2 h, 85-96%; e) TBAF (3.0 equiv), THF, 23° C., 2 h, quant.; j) R_{F}—PPh₃ (2.0 equiv), R_{F}-DIAD (2.0 equiv), PhMe, 23° C., 2 h, 50-85%; g) 1% NaOH in MeOH, reflux, 12 h, 80-90%; h) PS—SO₃H (5.0 equiv), MeOH, 50° C., 2 h, >90%; i) PS-IBX (3.0 equiv), CH₂Cl₂/few drops of DMSO, 23° C., 1-3 h (monitored by TLC), 50%; j) DMP (1.5 equiv), CH₂Cl₂, reflux, 4 h, 80-90%; k) 40% aq. HF in CH₃CN (1/10), 23° C., 3-6 h, 50-70%. DDQ=2,3-dichloro-5,6-dicyanobenzoquinone, DIAD=diisopropyl azodicarboxylate, DMF=N,N-dimethylformamide, DMP=Dess-Martin periodinane, DMSO=dimethylsulfoxide, EOM=ethoxymethyl, HMPA=hexamethylphosphoramide, IBX=2-iodoxybenzoic acid, LDA=lithium diisopropylamide, PS=polystyrene, TBAF=tetrabutylammonium fluoride, THF=tetrahydrofuran.

FIG. 8. a) DMDO (4.0 equiv), CH₃CN, 0° C. 1-2 h, 60-70%; b) CH₂N₂ (5-10 equiv), Et₂O, 23° C., 3-12 h, 50-60%; c) RNH₂OH.HCl (10.0 equiv), pyridine, 40° C., 12 h, >50%. DMDO=dimethyldioxirane.

FIG. 9. IC₅₀ of selected library members against a panel of 19 kinases.

FIG. 10. Residual activity as a percentage of control for 31 of the 46 putative kinase inhibited by two a resorcylide containing an alkane (1ath) or ether (1bgi) at the benzylic position.

FIG. 11. Residual activity as a percentage of control for mutation of kinases.

FIG. 12. Comparison of cellular efficacy between 1agh, 1aej and known approved drug on RENCA renal carcinoma (Sunitinib and Sorafenib).

FIG. 13. Divergent synthesis of compounds 3 and 5.

FIG. 14. Activity of different RALs in a RENCA cellular proliferation assay.

FIG. 15. In vivo efficacy. Group 1: vehicle (n=12/10); Group 2: sunitinib, 20 mg, q1d (n=12); Group 3: 3, 20 mg, q1d (n=12/11); Group 4: 3, 20 mg, q2d (n=12); Group 5: 5, 20 mg, q1d (n=12/11). A: In vivo luciferase activity (Photons/s); B: Primary tumor volume (mm³); C: Primary tumor weight (g). Data are displayed as means+/−SEM. The inset shows the individual data points together with their corresponding mean values.

FIG. 16. Metases of to the lung. Group 1: vehicule (n=12/10); Group 2: sunitinib, 20 mg, q1d (n=12); Group 3: 3, 20 mg, q1d (n=12/11); Group 4: 3, 20 mg, q2d (n=12); Group 5: 5, 20 mg, q1d (n=12/11). A: Number of lung metastases Data are displayed as means+/−SEM. The inset shows the individual data points together with their corresponding mean values. B: Lung luciferase activity (LU/μg protein). Data are displayed as means+/−SEM. The inset shows the individual data points together with their corresponding mean values.

FIG. 17. Tumor vasculature histology (CD-31 staining) for vehicule group, group 3 (3, 20 mg, q1d); group 5: (5, 20 mg, q1d).

FIG. 18. Key disconnections for the synthesis of 5-fluoro-cis-enone resorcylides.

FIG. 19. Synthesis of fluoroenones 1 and 2. A: Synthesis of key intermediate 14 from ester 5. Reagents and conditions: a) TBDPSC1 (1.1 equiv), imidazole (2.0 equiv), CH₂Cl₂, 238C, 13 h, 90%; b) Dibal-H (1.1 equiv), PhMe, _{—}788C, 1 h, 85%; c) 1. (EtO)2POCHFCO2Et, nBuLi, THF, 0 8C, 1 h; 2. 6 (1.0 equiv), 0!238C, 12 h, 83%; d) Dibal-H (2.5 equiv), CH₂Cl₂, 0!238C, 85%; e) (COCl)2 (1.5 equiv), DMSO (2.45 equiv), Et3N (4.0 equiv), CH2Cl2, 1 h, 98%; f) 1. PMBO(CH2)2CCH (2.1 equiv), nBuLi (2.0 equiv), THF, _{—}788C, 1 h; 2. 7 (1.0 equiv), _{—}788C, 1 h, 62%; g) H2, Pd/CaCO3, MeOH, 238C, 25 min, 97%; h) BzCl (1.5 equiv), pyridine (2.5 equiv), CH2Cl2, 0!238C, 14 h, 91%; i) DDQ (1.2 equiv), CH₂Cl₂/H₂O, 238C, 3 h, 92%; j) PPh3 (1.8 equiv), imidazole (3.0 equiv), I2, CH2Cl2, 238C, 1.5 h, 90%; k) OSO4 (1%), NMO.H2O (2.0 equiv), THF, 238C, 15 h, 78%; 1) 2-methoxy propene (1.5 equiv), PPTS (0.1 equiv), CH2Cl2, 238C, 1 h, 93%. Abbreviations: Bz, benzoyl; Dibal-H, diisobutylaluminium hydride; DDQ, 2,3-dichloro-5,6-dicyanobenzoquinone; NMO, N-methylmorpholine N-oxide; PPTS, pyridinium p-tolenesulfonate; TBDPS, tert-butyldiphenylsilyl. B: Synthesis of fluoroenones 1 and 2 from key intermediate 14. Reagents and conditions: a) 1. LDA (2.0 equiv), THF/HMPA, _{—}788C, 10 min; 2. 14 (1.0 equiv), THF, _{—}788C, 20 min, 80%; 3. H2O2 (2.0 equiv), THF, 238C, 3 h, 92%; b) K2CO3 (2.0 equiv), 14 (1.0 equiv), DMF, 80 8C, 12 h, 99%; c) TBAF (10 equiv), THF, 238C, 48-56 h; d) PPh3 (2.0 equiv), DIAD (2.0 equiv), PhMe, 238C, 2-4 h, 70-74% (two steps); e) 1% NaOH in MeOH, 508C, 2-2.5 h, 69-76%; f) DMP (1.5 equiv), CH2Cl2, 238C, 14 h, 80-88%; g) 40% aq HF in CH3CN (1:10), 23 8C, 6.5 h, 65%; h) PS-SO3H (10 equiv), MeOH, 23 8C, 9 h, 70%. Abbreviations: DIAD, diisopropyl azodicarboxylate; LDA, lithiumdiisopropylamide; HMPA, hexamethylphosphoramide; DMF, N,N-dimethylformamide; DMP, Dess-Martin periodinane; TBAF, tetrabutylammoniumfluoride; PS, polystyrene supported.

FIG. 20. Inhibition of VEGF-R2 and KIT.

FIG. 21. Cellular inhibition of VEGFR2 by a) LL-Z1640-2 (IC50=6.5 nm), b) fluoroenone 1 (IC₅₀=17 nM) and c) fluoroenone 2 (IC₅₀=70 nM). Assays were performed in triplicate.

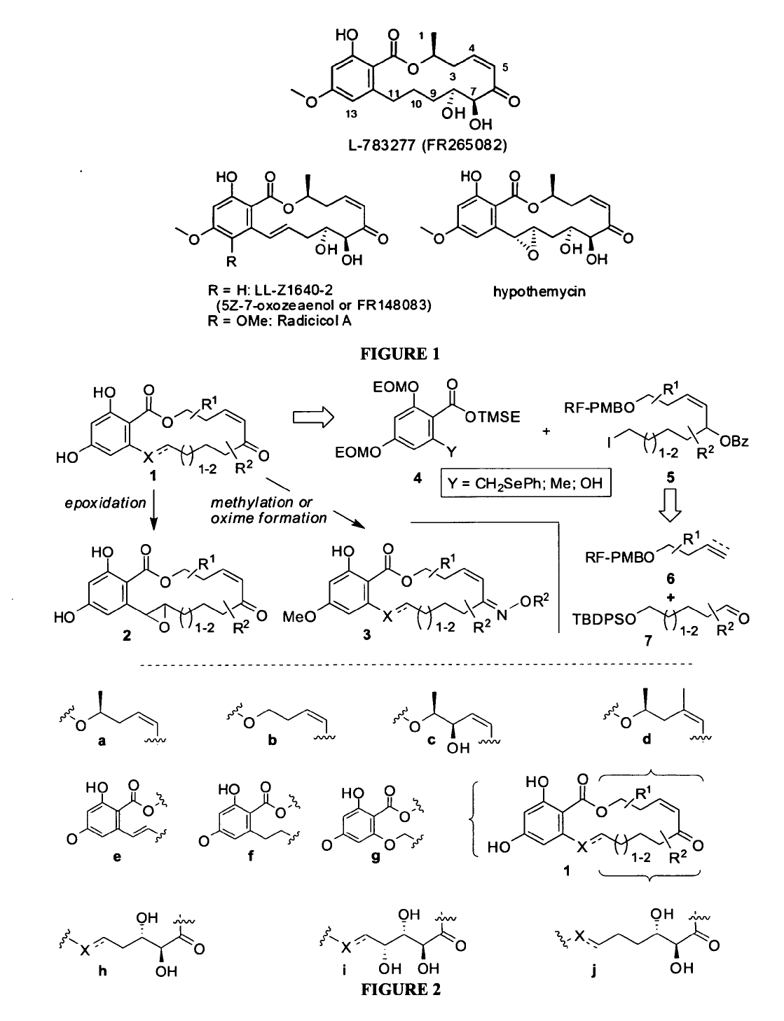

### DETAILED DESCRIPTION OF THE INVENTION

The present invention provides kinase inhibitors with improved potency and selectivity. The vascular endothelial growth factor receptors (VEGFRs) and platelet-derived growth factor receptors (PDGFRs) were predominantly the most inhibited kinases by the provided kinase inhibitors.

A broader profile against 359 kinases (excluding mutants) revealed a pattern similar to that of sunitinib (SU11248) which is generally considered a multitarget receptor kinase inhibitor of VEGFRs and PDGFRs. Compounds of Formula I were good inhibitors of VEGFRs in vitro, and inhibited tumor growth in vivo with comparable efficacy to sunitinib, an FDA-approved VEGFRs inhibitor.

Therefore, compounds of Formula I, as well as compounds of Formula II, IIa, IIb and/or III and any compound specifically disclosed herein, are of therapeutic interest for treating cancer. Indeed, multiple strategies for inhibiting the VEGF pathway have been shown to hinder tumor growth and the recent approval of small molecule inhibitors (sorafenib and sunitinib) as well as neutralizing antibodies (bevacizumab) targeting the VEGF receptors have clinically validated this strategy.

However, enduring clinical response are rare and there is mounting evidence that antiangiogenic therapy, while delaying primary tumor growth, can concomitantly promote tumor adaptation and progression to stages of greater malignancy with heighten invasiveness and distant metastasis. It has recently been shown that short-term sunitinib treatment resulted in accelerated metastasis in a mice model. However, most preclinical studies focus on primary tumor growth with less attention on metastasis. While sunitinib and sorafenib are very effective inhibitors of the angiogenic pathways and primary tumor growth, the inventors sought to evaluate resorcylic acids, as the irreversible inhibition of the target kinases may provide different outcome. Several programs to develop irreversible kinase inhibitors have already progressed to the clinic.

In addition, while compounds of Formula III did promote lung metastasis in an in vivo model to a similar extent as sunitinib, compounds of Formula II strongly inhibited lung metastasis. Therefore, compounds of Formula II are of a great therapeutic interest.

### Compounds

The present invention relates to a compound of formula I, a tautomer or pharmaceutically acceptable salt, solvate, or ester thereof,

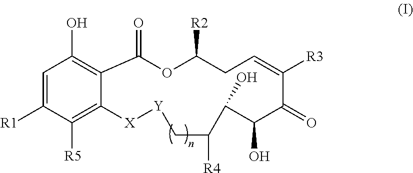

wherein,

n is 0 or 1;

R1 is selected from the group consisting of hydrogen, halogen, cyano, —OR, —NRR′, —NRS(O)R′, —NRS(O₂)R′, —SR, —S(O)R, —S(O₂)R, —OC(O)R, —C(O)R, —C(O)OR, —NRC(O)R′, —C(O)NRR′, —OC(O)OR, aliphatic, heteroaliphatic, acyl, aryl, heteroaryl, alkylaryl, arylalkyl, alkylheteroaryl and heteroarylalkyl;

R2 is hydrogen, or a C₁-C₅ alkyl;

R3 is hydrogen or halogen;

R4 is hydrogen or hydroxyl;

R5 is hydrogen, halogen, OR, NHR, NH—COR, R being hydrogen, or a C₁-C₅ alkyl;

X—Y is CH═CH, CH₂—CH₂, O—CH₂, CH₂—O, S—CH₂, or CH₂—S, and,

provided that the compound is not a compound of Formula I with

n is 0, R1 is hydroxyl, R3, R4 and R5 are hydrogen, R2 is methyl, X—Y is O—CH₂ or CH₂—S; or

n is 0, R1 is hydroxyl, R4 and R5 are hydrogen, R2 is methyl, R3 is fluorine, X—Y is O—CH₂; or

n is 0, R¹ is methoxy, R2 is methyl, R3 is fluorine, R4 and R5 are hydrogen, X—Y is CH₂—S.

By “C₁-C₅ alkyl” is intended a saturated alkyl, in particular methyl, ethyl, propyl, isopropyl, butyl, isobutyl, tert-butyl, pentyl, and the other isomeric forms thereof.

Preferably, the C₁-C₅ alkyl of Formula I is a C₁-C₃ alkyl. By “C₁-C₃ alkyl” is intended methyl, ethyl, propyl, and isopropyl.

By “halogen” is intended an halogen selected from bromine, fluorine, iodine and chlorine, more preferably fluorine or chlorine.

By “aliphatic” is intended straight-chain, branched or cyclic C₁-C₁₂ hydrocarbons which are completely saturated or which contain one or more units of unsaturation but which are not aromatic.

By “heteroaliphatic” as used herein is intended to have its customary meaning in the art and includes an aliphatic group substituted with one or more atoms other than carbon or hydrogen in the aliphatic chain, nonlimiting examples of which are nitrogen, oxygen, sulfur, phosphorus, silicon or boron.

The term “tautomer” as used herein refers to alternate structures which are recognized in the art to be in equilibrium with the depicted structure. For example, the enol structure below is a tautomer of the ketone structure and recognized to be in equilibrium with the ketone structure.

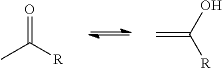

As used herein, the term “solvate” or “pharmaceutically acceptable solvate,” is a solvate formed from the association of one or more solvent molecules to one or more. molecules of a compound. of any one of formulae I, II, IIa, IIb, III or the compounds specifically disclosed herein. The term solvate includes hydrates (e.g., hemi-hydrate, mono-hydrate, dihydrate, trihydrate, tetrahydrate, and the like).

The term “aryl” as used herein is intended to have its customary meaning in the art, and includes any stable monocyclic, bicyclic, or tricyclic carbon ring, wherein at least one ring is aromatic, and especially phenyl, biphenyl, or naphthyl.

The term “aralkyl” or “arylalkyl” refers to an aryl group substituted with an alkyl substituent or linked to the molecule through an alkyl group as defined above.

The term “acyl” includes a group of the formula C(O)Rx, wherein Rx is an straight, branched, or cyclic alkyl.

The term “heterocycle”, “heterocyclyl”, or “heterocyclic” as used herein includes non-aromatic ring systems having four to fourteen members, preferably five to ten, in which one or more ring carbons, preferably one to four, are each replaced by a heteroatom.

Based on structure-activity relationship, it has been determined that R1 can include a high variety of groups, without changing the kinase inhibitory activity of the compound. Indeed, this substituent is pointing towards the solvent and only provides marginal changes in enzyme selectivity or affinity. On the other hand, a variety of substituent can be incorporated at that position to enhance the pharmacological properties at this position. For example, enzymatically stable substituent such as an alkyl ether may be used to reduce the metabolic clearance by inhibiting the glucuronidation and include a morpholino group to enhance aqueous solubility. Analogously, an aniline substituent provides a protonable group which can enhance aqueous solubility without affecting enzyme selectivity of affinity.

In a preferred embodiment, R1 is selected from the group consisting of —OR, —NHR and —SR. Preferably, R is selected from hydrogen, methyl, ethyl and isopropyl, more preferably from hydrogen or methyl.

Preferably, R3 is hydrogen or fluorine.

Preferably, R5 is hydrogen or halogen. Preferably, R5 is hydrogen or chlorine. In particular, when n is 0, R5 is preferably hydrogen.

Preferably, the compound of Formula I has one or several (2, 3, 4, 5 or all) of the following features:
n is 0 or 1; and/or
R1 is —OR, —SR or —NHR and R is selected from hydrogen, methyl, ethyl and isopropyl, preferably R1 is —OR, more preferably R1 is hydroxyl or methoxy; and/or
R2 is selected from hydrogen, methyl, ethyl and isopropyl, more preferably from hydrogen or methyl, still more preferably is a methyl; and/or
R3 is hydrogen or fluorine, preferably hydrogen; and/or
R4 is hydrogen or hydroxyl; and/or
R5 is hydrogen; and/or
X—Y is selected from CH═CH, CH₂—CH₂, O—CH₂, CH₂—O, S—CH₂, and CH₂—S, preferably from CH═CH, CH₂—CH₂, and O—CH₂.

In a first embodiment, the present invention relates to a compound of formula II, a tautomer or pharmaceutically acceptable salt, solvate, or ester thereof,

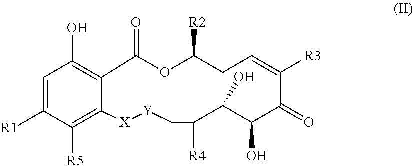

R1, R2, R3, R4, R5 and X—Y being as defined in Formula I.

More specifically, the present invention may relate to a compound of formula IIa, a tautomer or pharmaceutically acceptable salt, solvate, or ester thereof,

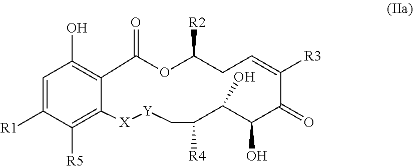

R1, R2, R3, R5 and X—Y being as defined in Formula I and R4 is hydroxy.

Alternatively, the present invention may relate to a compound of formula IIb, a tautomer or pharmaceutically acceptable salt, solvate, or ester thereof,

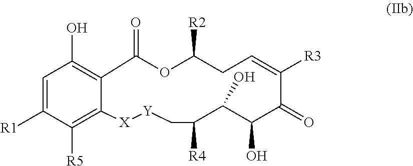

R1, R2, R3, R5 and X—Y being as defined in Formula I and R4 is hydroxy.

Preferably, the compound is of Formula II, IIa or IIb has one or several (2, 3, 4, 5 or all) of the following features:
R1 is —OR and R is selected from hydrogen, methyl, ethyl and isopropyl, more preferably R1 is hydroxyl or methoxy; and/or
R2 is selected from hydrogen, methyl, ethyl and isopropyl, more preferably from hydrogen or methyl, still more preferably is a methyl; and/or
R3 is hydrogen or fluorine, preferably hydrogen; and/or
R4 is hydrogen or hydroxyl, preferably hydrogen; and/or
R5 is hydrogen; and/or
X—Y is selected from CH═CH, CH₂—CH₂, O—CH₂, CH₂—O, S—CH, and CH—S, preferably from CH═CH, CH₂—CH₂, and O—CH₂.

In a particular preferred embodiment, the compound of Formula II does not have concomitantly a hydrogen for R2 and O—CH₂ for X—Y.

In a preferred embodiment, the compound of Formula II, IIa or IIb has one or several (2, 3, 4, 5 or all) of the following features:
R1 is hydroxyl or methoxy; and/or
R2 is hydrogen or methyl; and/or
R3 is hydrogen; and/or
R4 is hydrogen; and/or
R5 is hydrogen; and/or
X—Y is selected from CH═CH, CH₂—CH₂, and O—CH₂;

provided that the compound of Formula II, IIa or IIb does not have concomitantly a hydrogen for R2 and O—CH₂ for X—Y.

In another particular preferred embodiment, the compound of Formula II, IIa or IIb presents CH₂—CH₂ for X—Y.

In a particular embodiment, the compound of Formula II, IIa or IIb presents CH₂—CH₂ for X—Y and has one or several of the following features:
R1 is hydroxyl or methoxy; and/or
R2 is hydrogen or methyl; and/or
R3 is hydrogen; and/or
R4 is hydrogen; and/or
R5 is hydrogen.

In another particular embodiment, the compound of Formula II, IIa or IIb presents R3, R4 and R5 being hydrogen, R1 being hydroxyl or methoxy, and R2 being hydrogen or methyl and X—Y being selected from CH═CH, CH₂—CH₂, and O—CH₂. Preferably, when R2 is hydrogen, X—Y is CH═CH or CH₂—CH₂, more preferably is CH₂—CH₂. Alternatively, when R2 is methyl, X—Y is selected from CH═CH, CH₂—CH₂, and O—CH₂. More preferably, R1 is hydroxyl.

In a particularly preferred embodiment, the compound of Formula II, IIa or IIb has all the above mentioned features. Accordingly, the compound of Formula II, IIa or IIb has R1 being hydroxyl or methoxy, R2 being hydrogen or methyl, R3, R4 and R5 being hydrogen, and X—Y being selected from CH═CH, CH₂—CH₂, O—CH₂, provided that the compound does not have concomitantly a hydrogen for R2 and O—CH₂ for X—Y. Therefore, a particular embodiment of the invention relates to a compound of Formula II, IIa or IIb having R1 being hydroxyl or methoxy, R2, R3, R4 and R5 being hydrogen, and X—Y being CH═CH or CH₂—CH₂, more preferably being CH₂—CH₂. In an alternative embodiment, the invention relates to a compound of Formula II, IIa or IIb having R1 being hydroxyl or methoxy, R2 being methyl, R3, R4 and R5 being hydrogen, and X—Y being selected from CH═CH, CH₂—CH₂, and O—CH₂.

The invention relates in particular to one or several of the following compounds of Formula II, IIa or IIb:
a compound of formula II, wherein R1 is hydroxyl, R3, R4 and R5 are hydrogen, R2 is methyl, and X—Y is CH═CH;

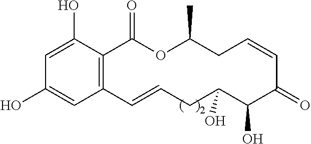

(also called herein 1aej or RAL 5)
a compound of formula II, wherein R1 is hydroxyl, R3, R4 and R5 are hydrogen, R2 is methyl, and X—Y is CH₂—CH₂;

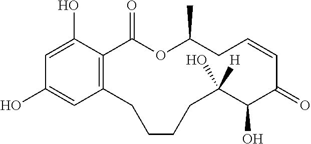 [SMILES: C[C@H]1C/C=C\C(=O)[C@@H](O)[C@@H](O)CCCCc2cc(O)cc(O)c2C(=O)O1]

(also called herein 1afj or RAL 1)
a compound of formula II, wherein R1 is hydroxyl, R3, R4 and R5 are hydrogen, R2 is methyl, and X—Y is O—CH₂;

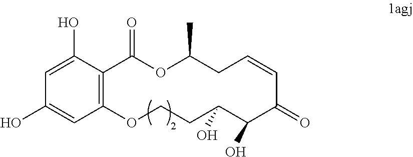

a compound of formula II, wherein R1 is hydroxyl, R2, R3, R4 and R5 are hydrogen, and X—Y is CH₂—CH₂;

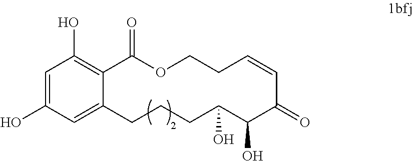

a compound of formula II, wherein R3, R4 and R5 are hydrogen, R1 is methoxy, and R2 is methyl, and X—Y is CH═CH;

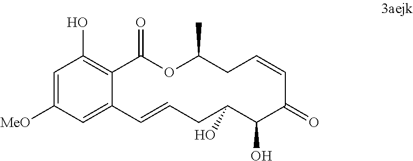 [SMILES: COc1cc(O)c2c(c1)/C=C/C[C@H](O)[C@H](O)C(=O)/C=C\C[C@H](C)OC2=O]

a compound of formula II, wherein R3, R4 and R5 are hydrogen, R1 is methoxy, and R2 is methyl, and X—Y is CH₂—CH₂;

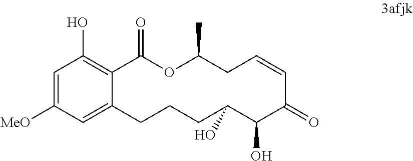 [SMILES: COc1cc(O)c2c(c1)CCC[C@H](O)[C@H](O)C(=O)/C=C\C[C@H](C)OC2=O]

a compound of formula II, wherein R2, R3, R4 and R5 are hydrogen, R1 is methoxy, and X—Y is CH₂—CH₂;

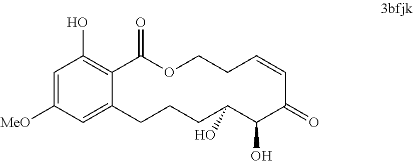 [SMILES: COc1cc(O)c2c(c1)CCC[C@H](O)[C@H](O)C(=O)/C=C\CCOC2=O]

In a second preferred embodiment, the present invention relates to a compound of formula III, a tautomer or pharmaceutically acceptable salt, solvate, or ester thereof,

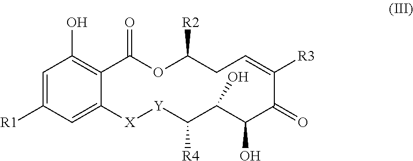

R1, R2, R3, R4, and X—Y being as defined in Formula I.

Preferably, the compound is of Formula III with R2 being selected from hydrogen, methyl, ethyl and isopropyl, more preferably from hydrogen and methyl, still more preferably being a methyl.

In particular, the compound of Formula III may have one or several (2, 3, 4, 5 or all) of the following features:
R1 is —OR and R is selected from hydrogen, methyl, ethyl and isopropyl, more preferably R1 is hydroxyl or methoxy; and/or
R2 is selected from hydrogen, methyl, ethyl and isopropyl, more preferably from hydrogen and methyl, still more preferably is a methyl; and/or
R3 is hydrogen or fluorine, preferably hydrogen; and/or
R4 is hydrogen or hydroxyl; and/or
X—Y is selected from CH═CH, CH₂—CH₂, O—CH₂, CH₂—O, S—CH₂, and CH₂—S, preferably from CH═CH, CH₂—CH₂, and O—CH₂.

More preferably, the compound is of Formula III may have one or several (2, 3, 4, 5 or all) of the following features:
R1 is hydroxyl; and/or
R2 is hydrogen or methyl, preferably methyl; and/or
R3 is hydrogen or fluorine, preferably hydrogen; and/or
R4 is hydrogen or hydroxyl; and/or
X—Y is selected from CH═CH, CH₂—CH₂, O—CH₂.

In a preferred embodiment, compounds of Formula III with R2 being methyl, ethyl and isopropyl, preferably methyl, and with R4 being hydroxyl are contemplated as they present a significant gain of activity.

In a particular embodiment of the compounds of Formula III, when R4 is H, then X—Y is preferably not O—CH₂ or CH₂—S. Preferably, X—Y is CH═CH or CH₂—CH₂.

The invention relates in particular to one or several of the following compounds of Formula III:
a compound of formula III, wherein R1 is hydroxyl, R3 and R4 are hydrogen, R2 is methyl, and X—Y is CH₂—CH₂;

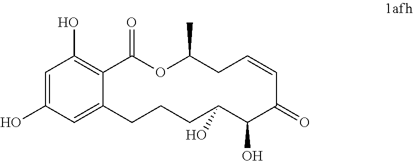 [SMILES: C[C@H]1C/C=C\C(=O)[C@@H](O)[C@@H](O)CCCc2cc(O)cc(O)c2C(=O)O1]

a compound of formula III, wherein R1 is hydroxyl, R3 is hydrogen, R2 is methyl, R4 is hydroxyl, and X—Y is O—CH₂;

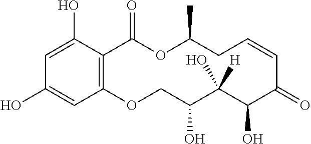 [SMILES: C[C@H]1C/C=C\C(=O)[C@@H](O)[C@@H](O)[C@H](O)COc2cc(O)cc(O)c2C(=O)O1]

### RAL 4 ou 1agi

a compound of formula III, wherein R1 is hydroxyl, R2, R3 and R4 are hydrogen, and X—Y is O—CH₂;

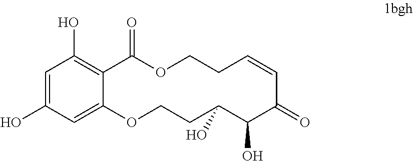 [SMILES: O=C1OCC/C=C\C(=O)[C@@H](O)[C@@H](O)CCOc2cc(O)cc(O)c21]

a compound of formula III, wherein R1 is hydroxyl, R2 and R3 are hydrogen, R4 is hydroxyl, and X—Y is O—CH₂;

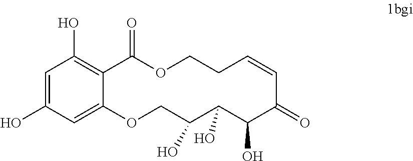 [SMILES: O=C1OCC/C=C\C(=O)[C@@H](O)[C@@H](O)[C@H](O)COc2cc(O)cc(O)c21]

a compound of formula III, wherein R1 is hydroxyl, R4 is hydrogen, R2 is methyl, R3 is fluorine, and X—Y is CH═CH;

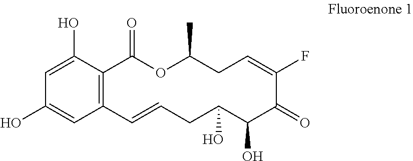 [SMILES: C[C@H]1C/C=C(/F)C(=O)[C@@H](O)[C@@H](O)C/C=C/c2cc(O)cc(O)c2C(=O)O1]

The term “pharmaceutically acceptable salt” is used throughout the specification to describe any pharmaceutically acceptable form (such as a salt, an ester, a phosphate ester, salt of an ester or a related. group) of a compound which, upon administration to a patient, provides the compound described in the specification. In cases where compounds are sufficiently basic or acidic to form stable nontoxic acid or base salts, administration of the compounds as salts may be appropriate. The term pharmaceutically acceptable salts or complexes refers to salts or complexes that retain the desired biological activity of the compounds of the present invention and exhibit minimal undesired toxicological effects.

No limiting examples of such salts are (a) acid addition salts formed with inorganic acids such as sulfate, nitrate, bicarbonate, and carbonate salts (for example, hydrochloric acid, hydrobromic acid, sulfuric acid, phosphoric acid, nitric acid, and the like), and salts formed with organic acids including tosylate, methanesulfonate, acetate, citrate, malonate, tartarate, succinate, benzoate, ascorbate, α-ketoglutarate, and α-glycerophosphate salts, such as acetic acid, oxalic acid, tartaric acid, succinic acid, malic acid, ascorbic acid, benzoic acid, tannic acid, pamoic, acid, alginic acid, polyglutamic acid, naphthalenesulfonic acid, naphthalenedisulfonic acid, and polygalcturonic acid, (b) base addition salts formed with metal cations such as zinc, calcium, bismuth, barium, magnesium, aluminum, copper, cobalt, nickel, cadmium, sodium, potassium, lithium and the like, or with a cation formed from ammonia, N,N-dibenzylethylenediamine, D-glucosamine, tetraethylammonium, or ethylenediamine, or (c) combinations of (a) and (h), e.g., a zinc tannate salt or the like. Also included in this definition are pharmaceutically acceptable quaternary salts known by those. skilled in the art, which specifically include the quaternary ammonium salt of the formula —NR⁺A, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Pharmaceutically acceptable salts may be obtained using standard procedures well known in the art, (hr example by reacting a sufficiently basic compound such as an amine with a suitable acid affording a physiologically acceptable anion.

Compounds of the present invention have chiral centers and may exist in and be isolated in optically active and racemic forms. The present invention encompasses any racemic optically-active, diastereomeric, polymorphic, or stereoisomeric form, or mixtures thereof, of a compound of the invention, which possess the useful properties described herein. It is understood that based on the number of asymmetric centers, a total number of 2^{B} possible stereochemical isomers is possible. The present invention includes all possible stereochemical configurations of the compounds.

In some embodiments, the stereochemistry of the compounds of the invention will retain the natural stereochemistry of the natural resorcylic acid lactone. In other embodiments, the stereochemical configuration will be different than that found in radicicol A. It will be understood that the stereochemical configuration of any substituent at an asymmetric carbon of the compounds can be in the R or S configuration independent of other substituents at other asymmetric centers in the compound.

In one embodiment, the compounds are prepared in optically active form by asymmetric synthesis using the processes described herein or synthetic transformations known to those skilled in the art.

Examples of methods to obtain optically active materials are known in the art, and include at least the following: i) physical separation of crystals; ii) simultaneous crystallization; iii) enzymatic resolutions; IV) enzymatic asymmetric synthesis; v) chemical asymmetric synthesis; vi) diastereomer separations; vii) first- and second-order asymmetric transformations; viii) kinetic resolutions; ix) enantiospecific synthesis from non-racemic precursors; x) chiral liquid chromatography; xi) chiral gas chromatography; xii) extraction with chiral solvents; or xiii) transport across chiral membranes.

Compounds as disclosed herein may be prepared by the one skilled in the art based on its general knowledge, on the teaching of WO 2008/002497 and on the teaching of the examples of the present application.

Formulations

The pharmaceutical compositions as disclosed herein may further comprise an additive. The additive may be selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

The pharmaceutical compositions as disclosed herein may comprise a pharmaceutically acceptable carrier that is suitable for oral, parenteral, inhalation, topical, or intradermal administration. Optionally, compounds can be administered subcutaneously, intravenously, intramuscularly, parenterally, orally, submucosally, by inhalation, transdermally via a slow release patch, or topically, in an effective dosage range to treat cancer.

The pharmaceutical compositions may further comprise an additional active ingredient. Preferably, the additional active ingredient is an antitumoral drug.

Uses

The present invention relates to a compound of Formula (I), (II), (IIa), (Iib) or (III) or any particular compound disclosed herein as a drug. In particular, the compound is for use for treating cancer. It also relates to the use of the compound for the preparation of a medicament for treating cancer. It finally relates to a method for treating cancer in a subject, comprising administering a therapeutic effective amount of the compound.

In a specific embodiment, the compound is selected from the group consisting of:
a compound of formula III, wherein R1 is hydroxyl, R3 and R4 are hydrogen, R2 is methyl, and X—Y is CH₂—CH₂;
a compound of formula III, wherein R1 is hydroxyl, R3 is hydrogen, R2 is methyl, R4 is hydroxyl, and X—Y is O—CH₂;
a compound of formula III, wherein R1 is hydroxyl, R2, R3 and R4 are hydrogen, and X—Y is O—CH₂;
a compound of formula III, wherein R1 is hydroxyl, R2 and R3 are hydrogen, R4 is hydroxyl, and X—Y is O—CH₂;
a compound of formula III, wherein R1 is hydroxyl, R4 is hydrogen, R2 is methyl, R3 is fluorine, and X—Y is CH═CH;
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and,
a compound of formula II, wherein R2, R3, R4 and R5 are hydrogen, R1 is methoxy and X—Y is CH₂—CH₂.

Preferably, the compound is selected from the group consisting of:
a compound of formula III, wherein R1 is hydroxyl, R3 and R4 are hydrogen, R2 is methyl, and X—Y is CH₂—CH₂;
a compound of formula III, wherein R1 is hydroxyl, R3 is hydrogen, R2 is methyl, R4 is hydroxyl, and X—Y is O—CH₂;
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, and R2 is methyl, and X—Y is CH₂—CH₂; and
a compound of formula II, wherein R2, R3, R4 and R5 are hydrogen, R1 is methoxy, and X—Y is CH₂—CH₂.

In a particularly preferred embodiment, the present invention relates to any of the above-disclosed compounds of Formula (II) for use in the treatment of cancer while preventing metastasis occurrence, to the use of any of the above-disclosed compounds for the preparation of a medicament for treating cancer while preventing metastasis occurrence, and to a method for treating cancer while preventing metastasis occurrence in a subject, comprising administering a therapeutic effective amount of any of the above-disclosed compounds to said subject.

In a specific embodiment, the compound is selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and,
a compound of formula II, wherein R2, R3, R4 and R5 are hydrogen, R1 is methoxy and X—Y is CH₂—CH₂.

More preferably, the compound is selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, and R2 is methyl, and X—Y is CH₂—CH₂; and
a compound of formula II, wherein R2, R3, R4 and R5 are hydrogen, R1 is methoxy, and X—Y is CH₂—CH₂.

Within the context of the invention, the term treatment denotes curative, symptomatic, and preventive treatment. Pharmaceutical compositions and preparations of the invention can be used in humans with existing cancer or tumor, including at early or late stages of progression of the cancer. The pharmaceutical compositions and preparations of the invention will not necessarily cure the patient who has the cancer but will delay or slow the progression or prevent further progression of the disease, ameliorating thereby the patients' condition. In particular, the pharmaceutical compositions and preparations of the invention reduce the development of tumors, reduce tumor burden, produce tumor regression in a mammalian host and/or prevent metastasis occurrence and cancer relapse. In treating the cancer, the pharmaceutical composition of the invention is administered in a therapeutically effective amount.

By “effective amount” it is meant the quantity of the pharmaceutical composition of the invention which prevents, removes or reduces the deleterious effects of cancer in mammals, including humans. It is understood that the administered dose may be adapted by those skilled in the art according to the patient, the pathology, the mode of administration, etc.

An effective dose can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

The cancer may be a solid cancer or a hematopoietic cancer. The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), chronic lymphocytic leukemia, mastocytosis and any symptom associated with mastocytosis.

The compounds of the invention may be also administered in specific, measured amounts in the form of an aqueous suspension by use of a pump spray bottle. The aqueous suspension compositions of the present invention may be prepared by admixing the compounds with water and other pharmaceutically acceptable excipients. The aqueous suspension compositions according to the present invention may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycenn and propylene glycol, acids, bases or buffer substances for adjusting the pH, e g, citric acid, sodium citrate, phosphoric acid, sodium phospate as well as mixtures of citrate and phosphate buffers; surfactants, e.g. Polysorbate 80, and antimicrobial preservatives, e.g., benzalkomum chloride, phenylethyl alcohol and potassium sorbate.

Typical systemic dosages for all of the herein described conditions are those ranging from 0.01 mg/kg to 1500 mg/kg of body weight per day as a single daily dose or divided daily doses. Preferred dosages for the described conditions range from 0.5-1500 mg per day. A more particularly preferred dosage for the desired conditions ranges from 5-750 mg per day. Typical dosages can also range from 0 01 to 1500, 0.02 to 1000, 0.2 to 500, 0.02 to 200, 0.05 to 100, 0.05 to 50, 0.075 to 50, 0 1 to 50, 0.5 to 50, 1 to 50, 2 to 50, 5 to 50, 10 to 50, 25 to 50, 25 to 75, 25 to 100, 100 to 150, or 150 or more mg/kg/day, as a single daily dose or divided daily doses In one embodiment, the compounds are given in doses of between about 1 to about 5, about 5 to about 10, about 10 to about 25 or about 25 to about 50 mg/kg. Typical dosages for topical application are those ranging from 0.001 to 100% by weight of the active compound

The compounds are conveniently administered in units of any suitable dosage form, including but not limited to one containing from about 7 to 3000 mg, from about 70 to 1400 mg, or from about 25 to 1000 mg of active ingredient per unit dosage form. For example, an oral dosage of from about 50 to 1000 mg is usually convenient, including in one or multiple dosage forms of 50, 100, 200, 250, 300, 400, 500, 600, 700, 800, 900 or 1000 mgs. Lower dosages may be preferable, for example, from about 10-100 or 1-50 mgs. Also contemplated are doses of 0 1-50 mg, 0.1-20 mgs, or 0.1-10 mgs. Furthermore, lower doses may be utilized in the case of administration by a non-oral route, as for example, by injection or inhalation.

Pharmaceutically acceptable earners that may be used in these pharmaceutical compositions are generally known in the art. They include, but are not limited to, ion exchangers, alumina, aluminum stearate, lecithin, serum proteins, such as human serum albumin, buffer substances such as phosphates, glycine, sorbic acid, potassium sorbate, partial glycende mixtures of saturated vegetable fatty acids, water, solvents, salts or electrolytes, such as protamine sulfate, disodium hydrogen phosphate, potassium hydrogen phosphate, sodium chloride, zinc salts, silicates, colloidal silica, magnesium tπsihcate, polyvinyl pyrrolidone, cellulose-based substances, polyethylene glycol, sodium carboxymethylcellulose, polyacrylates, waxes, oils, polyethylene-polyoxypropylene-block polymers, polyethylene glycol and wool fat. Pharmaceutically accepted vehicles can contain mixtures of more than one excipient in which the components and the ratios can be selected to optimize desired characteristics of the formulation including but not limited to shelf-life, stability, drug load, site of delivery, dissolution rate, self-emulsification, control of release rate and site of release, and metabolism.

Formulations can be prepared by a variety of techniques known in the art. Examples of formulation techniques can be found in literature publications and in texts such as “Water-insoluble drug formulation”, edited by Rong Liu, 2000, Interpharm Press

If administered intravenously, earners can be physiological saline, bacteriostatic water, Cremophor EL™ (BASF, Parsippany, N.J.) or phosphate buffered saline (PBS). Sterile injectable forms of the compositions of this invention may be aqueous or oleaginous suspension. These suspensions may be formulated according to techniques known in the art using suitable dispersing or wetting agents and suspending agents The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other surface-active emulsifying agents or bioavailability enhancers which are commonly used in the manufacture of pharmaceutically acceptable solid, liquid, or other dosage forms may also be used for the purposes of formulation.

One mode of administration of the active compound for systemic delivery is oral. Oral compositions will generally include an inert, diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a ghdant such as colloidal silicon dioxide a sweetening agent such as sucrose or saccharin; or a flavoring agent such as peppermint, methyl salicylate, or orange flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. The compound or its salts can be administered as a component of an elixir, suspension, syrup, wafer, chewing gum or the like. A syrup may contain, in addition to the active compounds, sucrose as a sweetening agent and certain preservatives, dyes and colorings and flavors.

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable earners. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container An aqueous solution of the compound is then introduced into the container. The container is then swirled by hand to free lipid material from the sides of the container and to disperse lipid aggregates, thereby forming the liposomal suspension.

Suitable vehicles or earners for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients, and stabilizers can be used. to prepare topical compositions. Examples of thickening agents include petrolatum, beeswax, xanthan gum, or polyethylene, humectants such as sorbitol, emollients such as mineral oil, lanolin and its derivatives, or squalene.

The pharmaceutical composition as disclosed herein may be used in combination with chemotherapy, radiotherapy, hormonal therapy, immunotherapy, or monoclonal antibody therapy.

Chemotherapy refers to a cancer therapeutic treatment using chemical or biochemical substances, in particular using one or several antineoplastic agents.

Radiotherapy includes, but is not limited to, y-rays, X-rays, and/or the directed delivery of radioisotopes to tumor cells. Other radiotherapies include microwaves and UV-irradiation. Other approaches to radiation therapy are also contemplated in the present invention.

The term “immunotherapy” refers to a cancer therapeutic treatment using the immune system to reject cancer. The therapeutic treatment stimulates the patient's immune system to attack the malignant tumor cells. It includes immunization of the patient with tumoral antigens (eg. by administering a cancer vaccine), in which case the patient's own immune system is trained to recognize tumor cells as targets to be destroyed, or administration of molecules stimulating the immune system such as cytokines, or administration of therapeutic antibodies as drugs, in which case the patient's immune system is recruited to destroy tumor cells by the therapeutic antibodies. In particular, antibodies are directed against specific antigens such as the unusual antigens that are presented on the surfaces of tumors. As illustrating example, one can cite Trastuzumab or Herceptin antibody which is directed against HER2 and approved by FDA for treating breast cancer.

The term “monoclonal antibody therapy” refers to any antibody that functions to deplete tumor cells in a patient. In particular, therapeutic antibodies specifically bind to antigens present on the surface of the tumor cells, e.g. tumor specific antigens present predominantly or exclusively on tumor cells. Alternatively, therapeutic antibodies may also prevent tumor growth by blocking specific cell receptors.

The term “hormone therapy” or “hormonal therapy” refers to a cancer treatment having for purpose to block, add or remove hormones. For instance, in breast cancer, the female hormones estrogen and progesterone can promote the growth of some breast cancer cells. So in these patients, hormone therapy is given to block estrogen and a non-exhaustive list commonly used drugs includes: Tamoxifen, Fareston, Arimidex, Aromasin, Femara, Zoladex/Lupron, Megace, and Halotestin.

### EXAMPLES

### Example 1

A library of resorcylic acid lactones (RAL) containing a cis-enone moiety targeting kinases bearing a cysteine residue within the ATP-binding pocket was prepared and evaluated against a panel of 19 kinases thus providing important structure-activity trends.

Two new analogues were then profiled for their selectivity against a panel of 402 kinases providing the broadest evaluation of this pharmacophores' selectivity.

We report here a library of over 50 analogues based on this resorcylic acid scaffold and the activity of a representative subset of 31 members was evaluated against a panel of 19 kinases. Two analogues were further evaluated against a panel of 402 kinases which includes 359 distinct kinases and 43 mutants.

Results and Discussion

The synthetic planning of the library was based on the previously developed chemistry (P. Y. Dakas, et al, Angew. Chem. Int. Ed. Engl. 2007, 46, 6899-6902; P. Y. Dakas, et al, Chemistry. 2009 15, 11490-7) leveraged on the use of fluorous tags to facilitate isolation of reaction product and to carry mixtures of products through a common synthetic pathway (D. P. Curran, Handbook of Fluorous Chemistry 2004, 101-127; Z. Luo, et al, Science 2001, 291, 1766-1769; W. Zhang, D. P. Curran, Tetrahedron 2006, 62, 11837-11865). Thus a library of the general structure 1 (FIG. 2) was envisioned to emanate from the coupling of the key fragment 5 bearing a fluorous tag encoding its structure and the different aromatic moieties 4 bearing either the selenoether or methyl or hydroxyl at position Y to obtain respectively an alkene, an alkane or phenolic ether at position X in the library 1. While we have shown that this coupling chemistry could be carried out on solid phase using a thioether linker at position Y of intermediate 4 to access the alkane and alkene functionality (P. Y. Dakas, et al, Chemistry. 2009 15, 11490-7), this strategy precluded the formation of phenolic ethers which were deemed interesting and thus motivated the exclusive use of the fluorous isolation technology for the purpose of the present library. Fragment 5 was foreseen to come from the coupling of fragment 6 and 7, fragment 6 being either an alkyne which would be reduced to the cis alkene with Lindlar catalyst or a cis-vinyl iodide. Of course, the general library 1 could be further elaborated by epoxidation of the benzylic alkene to obtain hypothemycin analogues 2 or by methylation of the more acidic phenol and/or oxime formation on the ketone (3). Our choice of the fragments R¹, R² and aryl substitutions was based on our preliminary structure activity data coupled to reported structure activity obtained by semi-synthesis of hypothemycin (B. R. Hearn, et al., ChemMedChem 2007, 2, 1598-1600) and available structural information (M. Ohori, et al., Biochem. Biophys. Res. Commun. 2007, 353, 633-637; G. Rastelli, et al., J. Struct. Biol. 2008, 164, 18-23). Clearly, the nature of the functionality at the benzylic position appeared to be important in dictating the selectivity based on the afore mentioned difference in activity between LL-Z1640-2 bearing an alkene and L-783277 bearing an alkane for ERK2 (M. Ohori, et al., Biochem. Biophys. Res. Commun. 2007, 353, 633-637) and MEK1 (A. Zhao, et al., J. Antibio. 1999, 52, 1086-1094; J. Ninomiya-Tsuji, et al., J. Biol. Chem. 2003, 278, 18485-18490). While this modification at the benzylic position may seem rather modest, preliminary modeling experiments have suggested fairly different conformational landscape for both compounds. We thus felt important to include both of these functionalities in the library. While the epoxide at the benzylic position imparts a similar conformation to an alkene, difference in dipoles may be significant. Alternatively, it appeared that a phenolic ether at that position should also provide significant difference providing a conformational profile more similar to the alkane but with different dipole moments. While structural information could rule out certain modifications, it should be noted that the crystal structures reported (M. Ohori, et al., Biochem. Biophys. Res. Commun. 2007, 353, 633-637; G. Rastelli, et al., J. Struct. Biol. 2008, 164, 18-23) are for the Michael adduct and the initial recognition event between the protein and the cis-enone may involve different macrocyclic and/or kinase conformations. The modifications which were envisioned for the library are shown in FIG. 2 with four different fragments for the ester moiety R¹ (a-d), three different functionalities at position X (e-g) and three different fragments for the lower part of the macrocycle R² (h-j). The chiral methyl substituent on the ester points towards the surface of β-pleaded sheets. Our prior investigation had shown that the R-chirality at that center dramatically attenuated activity, nevertheless, compounds lacking that methyl group (fragment b) may broaden specificity. An extra hydroxyl group at the adjacent position (fragment c) was deemed interesting as it would probe whether additional interactions may be achieved and whether the chiral substituent imparts an important conformational bias on the macrocycle. The same reasoning was applied to the addition of a hydroxyl group on the lower part of the macrocycle (R², fragment i). Modification of the alkene (fragment d) was seen as crucial in modulating the rate of conjugate addition (if the conjugate addition is catalyzed by a hydrogen bond to the carbonyl, the greater stability of a tertiary cation vs. a secondary cation may favored the reaction). Furthermore, having a methyl substituent at that position could alleviate the issues related to cis/trans isomerization. It is known that the trans-enones are significantly less active than the cis-enone (A. Zhao, et al., J. Antibio. 1999, 52, 1086-1094). The homoallylic diol appeared to be important for biological activity and it was already known that methylation of either led to significant reduction in activity (B. R. Hearn, et al., ChemMedChem 2007, 2, 1598-1600). This functionality was thus kept constant in fragments h-j. Our preliminary investigations had shown that compounds lacking the diol were indeed inactive as well as acyclic analogues. However, a larger macrocycle appeared interesting in providing subtle changes in conformation profiles and could be probed with fragment j. While each of these modifications can be rationalized as providing a potential benefit, the combination of several modifications may provide unanticipated synergistic benefits.

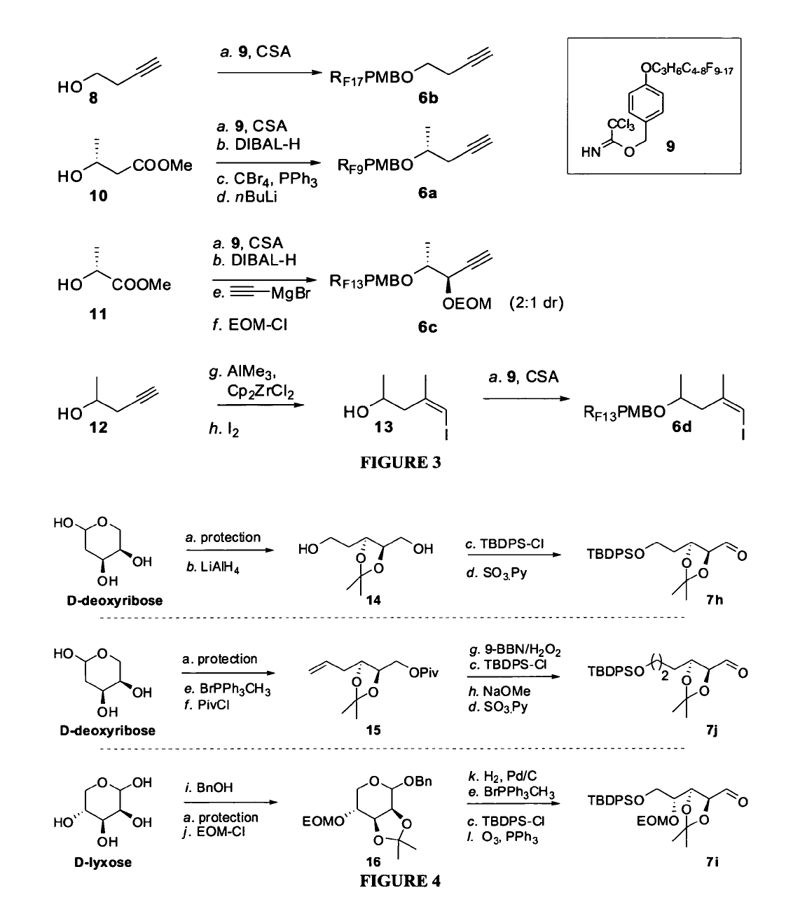

As shown in FIG. 3, fragments 6a-d were obtained in one to five steps via well established chemistry. The first step to obtain 6a-c involved a protection of the alcohols 8, 10 and 11 with the trichloroacetimidate of the fluorous-PMB (9) bearing different length of the fluorous tag encoding the structure of the starting alcohol. For 10 and 11, the alcohol protection was followed by a Dibal reduction and a Corey-Fuchs reaction or a Grignard addition of acetylene (2:1 dr) followed by EOM protection to afford 6a and 6c respectively. Compound 6d was prepared from the racemic 4-hydroxylpentyne using a known procedure (S. Ma, E.-i. Negishi, J. Org. Chem. 1997, 62, 784-785.) to access the Z-vinyl iodide 13 which was protected with the fluorous tagged PMB. The product of each reaction was isolated by flash chromatography on fluorous silica gel. In general, a 10 to 20 fold ratio of silica to crude product weight was used for the isolation. The elutions were carried out systematically using a three step gradient (7:3 MeOH:H₂O; 8:2 MeOH:H₂O and pure MeOH) and the product was collected from the MeOH fraction without further attempt to optimize individual isolation or recovered product from mixed fractions.

The synthesis of fragments 7h-j (FIG. 4) was leveraged on the naturally abundant chirality of deoxyribose and lyxose using well established methodology. Thus, D-deoxyribose was selectively protected with an acetonide (J. Barbat, et al., Carbohyd. Res. 1983, 116, 312-316) and reduced with LiAlH₄ to obtain diol 14 which was selectively protected on the less hindered alcohol with TBDPS-Cl and oxidized to aldehyde 7h. Starting with the same selective protection of ribose but engaging the resulting lactol in a Wittig olefination (X. Geng, S. J. Danishefsky, Org. Lett. 2004, 6, 413-416) rather than a reduction afforded alkene 15 after pivaloylation of the primary alcohol. A sequence involving a hydroboration, silyl protection, pivaloyl deprotection and oxidation afforded fragment 7j which has an additional carbon compared to 7h. The third fragment emanated from D-lyxose by protection of the anomeric position followed by selective acetonide protection of the cis-diol, and EOM-protection of the remaining hydroxyl group. Deprotection of the anomeric position and conversion of the lactol to an alkene allowed protection of the unique hydroxyl group with TBDPS-Cl followed by ozonolysis of the alkene to obtain fragment 7i. While a number of alternative strategies can be envisioned for each of these fragments, these procedures were found to be inexpensive, reliable and scalable.

The synthesis of key fragments 5 is shown in FIG. 5. Starting with a mixture of three fluorous tagged alkynes 6 which were deprotonated with nBu-Li and added to three vessels containing the different aldehydes 7 afforded three pools of 17, each containing three compounds labeled with a unique fluorous tag. Benzoyl protection of the alcohol followed by desilylation, hydrogenation over Lindlar catalyst and iodination afforded the three pools of three compounds 5a-c. All reactions were monitored by LC-MS and showed greater than 90% conversion. While the isolated yield of the product following fluorous isolation was not optimized, acceptable yields were generally obtained. The vinyl iodide 6d was transmetalated with tBuLi and similarly added to three different pools of aldehydes 7. A similar sequence of benzoylation, desilylation, and iodination afforded three pools of 5d.

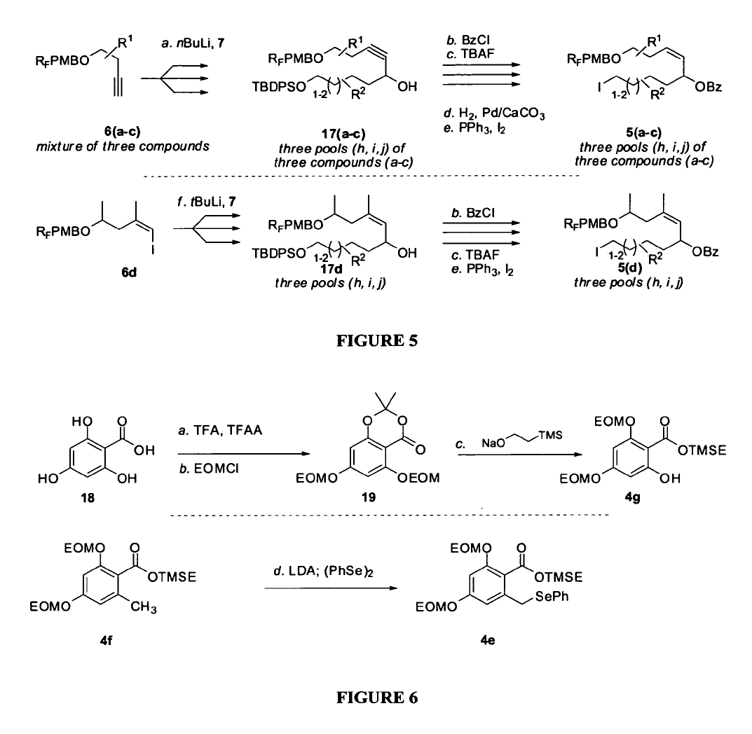

The aromatic fragments were obtained as shown in FIG. 6. The fragment 4g was obtained starting from 2,4,6-trihydroxybenzoicacid 18 by protection of the acid and ortho phenol with an acetonide (R. G. Dushin, S. J. Danishefsky, J. Am. Chem. Soc. 1992, 114, 655-659) followed by protection of the two remaining phenols with EOM-C1 to obtain 19 which was treated with the alkoxide of trimethylsilyl ethanol to yield 4g. Compound 4f was converted to 4e by deprotonation with LDA and reaction with diphenyl diselenide.

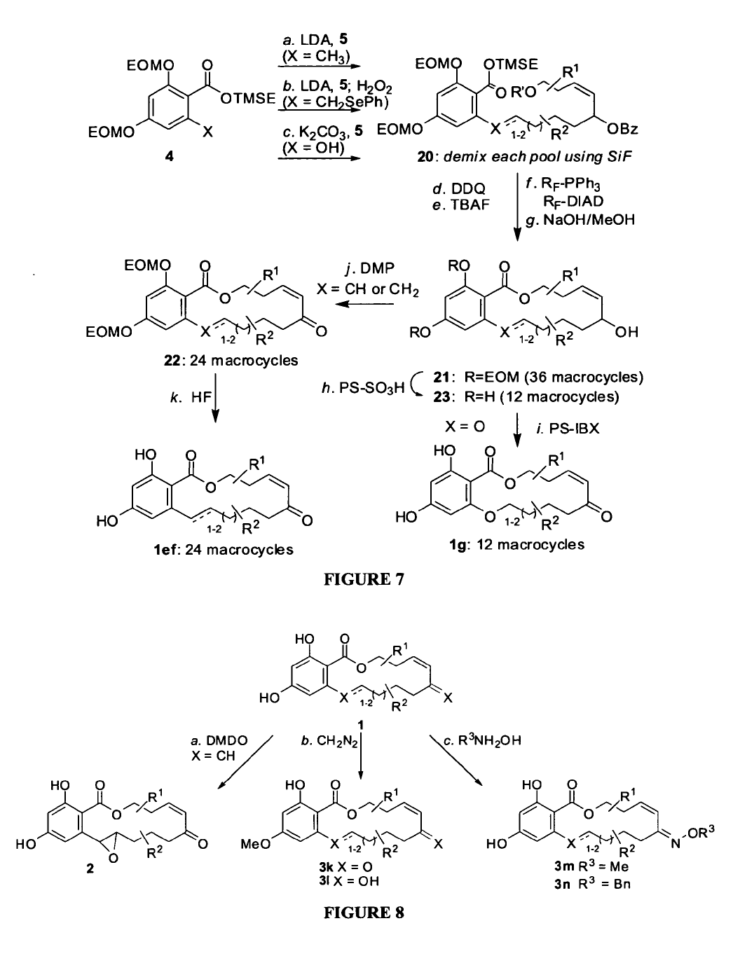

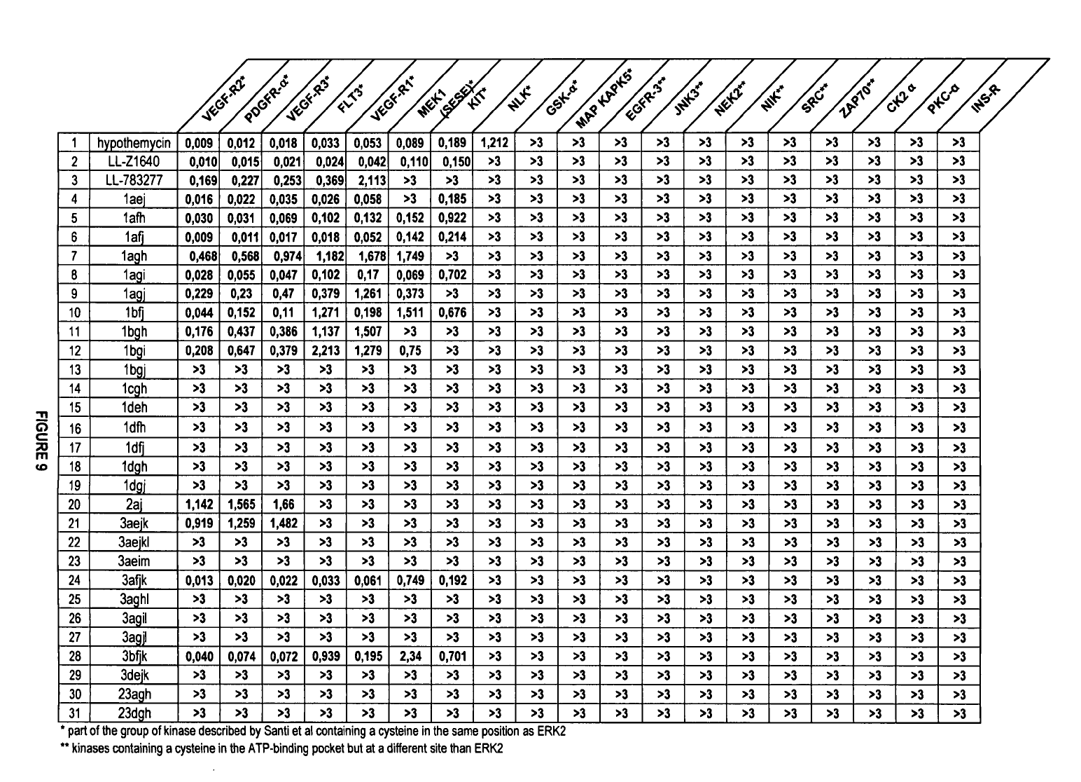

The library synthesis is shown in FIG. 7. Thus each aromatic fragment 4 was coupled to 4 pools of fragment 5. Analysis of the reaction mixtures by LC-MS indicated good to excellent conversion for all reactions, with the phenol couplings being systematically the highest yielding followed by the selenoether and alkyl couplings. Amongst the different pools of electrophiles, the pool containing fragment i with an additional EOM-protected hydroxyl adjacent to the iodide being displaced gave the lowest coupling efficiency. However, the desired coupled product was obtained in all cases. Each pool was demixed to resolve the individual components which were detagged (DDQ) and deprotected (TBAF) to be engaged in a macrolactonization. We had previously observed that these reactions could be performed at fairly high concentration (0.1 M) without dimerization or oligomerization. Treatment of each compound with fluorous-tagged PPh₃ and fluorous-tagged DIAD afford high yield in the lactonization (>85%) except for compounds containing the fragment c bearing a sterically more demanding alcohol for which 50% conversion was achieved. The allylic benzoate was removed in excellent yield for all macrocycles. We had previously noted that a selective allylic oxidation could be achieved in some cases but was unfortunately not general and depended on the relative geometry of the allylic alcohol (P. Y. Dakas, et al, Chemistry. 2009 15, 11490-7). In the cases of the macrocycles bearing an ether (X═O), the selective oxidation of the fully deprotected macrocycle was reliably achieved with polymer-bound IBX. On the other hand, for the oxidation of the macrocycles containing an alkane or alkene at the benzylic position, the oxidation was performed prior to deprotection using Dess-Martin periodinane in refluxing CH₂Cl₂ (the oxidation of one of the diastereoisomer is slower than the deprotected macrocycle and requires more stringent conditions). Removal of the acetonide and EOM using aqueous HF afforded the desired macrocycles in moderate to good yields. It should be noted that the reaction was never allowed to reach completion as prolonged reaction time led to product decomposition. Macrocycles 1ef bearing an EOM group on the para-phenol were also isolated from most reactions. All compounds were purified by preparative TLC. The macrocycles 1e could be further elaborated by epoxidation of the benzylic position using DMDO (P. Y. Dakas, et al, Chemistry. 2009 15, 11490-7). However, the lability of the benzylic epoxide makes isolation of the product challenging and pharmacologically undesirable. Macrocycles could be also further derivatized by selective methylation of the para-phenol with diazomethane. While the reaction was quite selective for the methylation of the para-phenol (3k and l, FIG. 8), to ensure completion of the reaction, these were performed with a large excess of diazomethane leading to the isolation of 10-40% of the bis-methylated product (not shown). The ketone could be converted to a methyl or benzyl oxime in high yield under standard conditions. In total, 51 macrocycles were isolated from these efforts.

The IC₅₀ of a subset of the library (28, containing at least one example of each modifications) was assayed against a panel of kinases (19) as representative of kinases bearing the adequately positioned cysteine residue (VGFR-R1-3, PDGF-Rα, FLT3, MEK1, KIT, GSKα, MAP KAPK5) kinases bearing a cysteine residue at a different position within the ATP binding pocket (EGFR-3, JNK3, NEK2, NIK, SRC, ZAP70) and kinases which do not bear a cysteine residue (PKCα, CK2α, INS-R). It should be noted that as these compounds are expected to be irreversible inhibitors, the measured IC₅₀ will depend on the procedure used and caution should be applied in comparing data from experiments performed under different conditions. To obtain a value which best reflects the rate of inactivation, the substrate and inhibitor were added simultaneously to the kinase solution. The data is shown in FIG. 9 (kinases have been ranked from the highest to least inhibited). As evident from FIG. 9, there is very little difference in relative selectivity of kinase inhibition throughout the library under these conditions. VEGF-R2 is the most highly inhibited kinase in this panel for all library members followed by PDGFR-α, VEGR-R3, Flt3, VEGF-R1, MEK1 SESE (which is a constitutively active form of MEK) and KIT. Overall, the trend of structure activity relationship which emerged from this screen is that the chiral methyl group at the ester position, while not essential, does provide higher activity (entry 6 vs. 10, 7 vs. 11 and 8 vs. 12). Substitution at the adjacent position with a hydroxyl group (entry 14 vs. 7) or at the β-position of the enone with a methyl abrogated activity (15-19). Position X of library 1 (FIG. 2), as suggested from the diverse natural products, is tolerant of modification and, an oxygen is also tolerated at that position. The trend of activity for the three natural products can be ranked as follows: alkene=epoxide>alkane. The ether in the macrocycle is generally comparable to the alkane. The two modifications of R² tested were beneficial in certain combinations. For example, the extra hydroxyl group present in fragment i does afford a significant (10 fold) gain of activity (8 vs. 7) in combination with fragment a. The same beneficial effect was not observed in the presence of fragment b (entry 12 vs. 11). The larger macrocycle (fragment j) was particularly beneficial in conjunction with the alkane moiety at the benzylic position (f). For example, entry 6, 10 and 25 are amongst the most potent compounds tested whereas a similar gain is not observed for X═CH or oxygen (entry 4, 9, 13). Generally, methylation of the phenol para to the ester had marginal changes in activity while oxime formation abolished activity. Interestingly, none of the alcohols 23 lacking the ketone showed significant inhibition attesting to the fairly low affinity of the macrocycle for the ATP-binding pocket and the importance of the Michael acceptor.

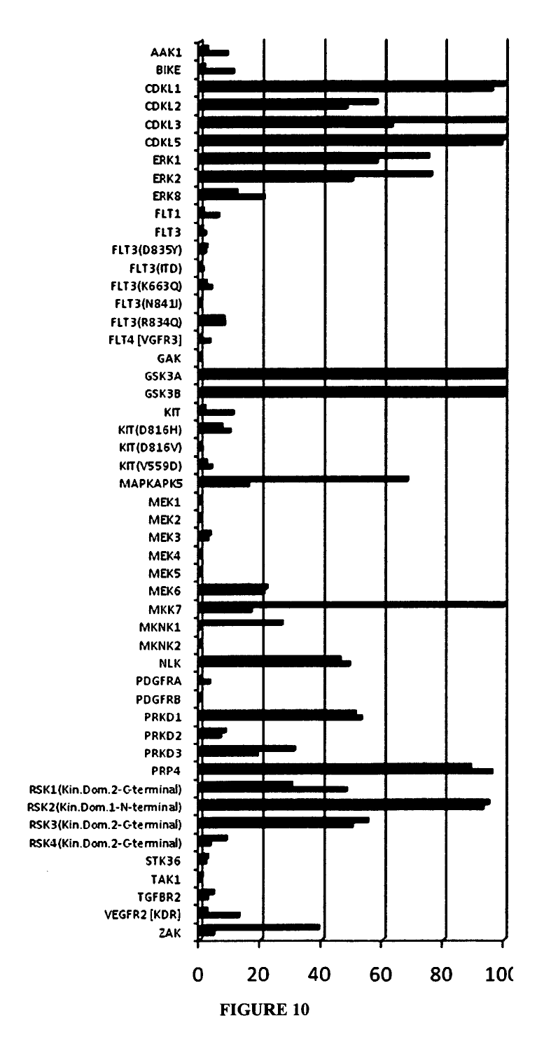

Two representative library members (1afh and 1bgi) were then evaluated in a larger panel of kinases (402) using the technology described by Lockhart et al. (M. A. Fabian, et al., Nat. Biotechnol. 2005, 23, 329-336; M. W. Karaman, et al., Nat. Biotechnol. 2008, 26, 127-132). In this panel, 31 kinases out of the 46 kinases bioinformatically identified by Santi and coworkers are present along with numerous other kinases bearing a cysteine residue at other positions within the ATP-binding pocket (J. Zhang, P. L. Yang, N. S. Gray, Nat. Rev. Cancer 2009, 9, 28-39). The two compounds were screened at 1 μM (i.e. well above their IC₅₀ concentration for the most potently inhibited kinases) to have a realistic perspective of their selectivity. Interestingly, only two kinase outside of the set of 46 kinases predicted by Santi et al showed significant inhibition (STK36 and PRKD2) attesting to the potential of exploiting cysteine residues within the ATP-binding pocket as selectivity filters (M. S. Cohen, et al., Science 2005, 308, 1318-1321). As shown in FIG. 10, 11 kinases out of 31 had more than 50% residual activity given this high concentration of 1afh (MEK4 had 0% residual activity at this concentration). The kinases which showed less than 10% residual activity relatively to control were: Flt1&3; GAK; KIT, MEK1,2,4; MKNK 1&2; PDGFRα&B; TAK1, TGFR2. Interestingly, some kinases in this group (CDKL5, ERK1, GSK3β, NIK, NLK, PRP4) were not inhibited. For 1bgi, 19 out of the 31 kinases had over 50% residual activity and MEK4 was also the most highly inhibited kinases having 1% residual activity. While 1bgi was overall less potent, it does appear to show subtle differences in reactivity compared to 1afh. For example, while both compounds have similar activity against GAK, 1bgi is ten times less active against Flt1 and PDGFRβ.

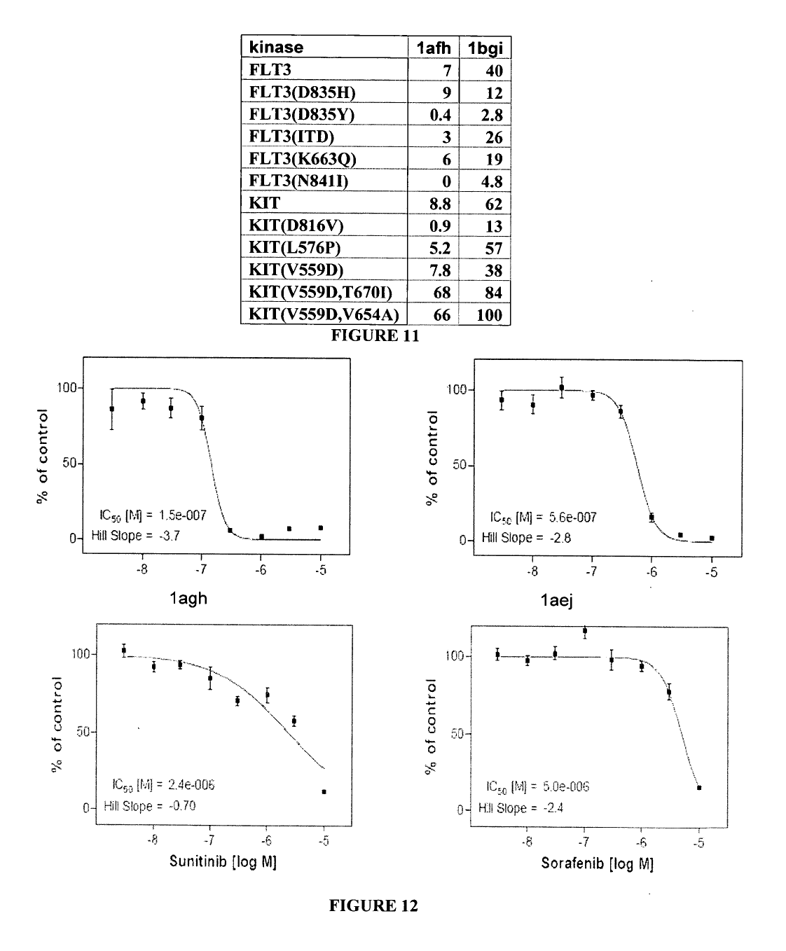

The two compounds were also evaluated against a series of mutations of Flt3 and KIT. It is known that several mutation in Flt3 lead to a gain of function or constitutive activity. An internal tandem duplication (ITD) in Flt3 (M. Nakao, et al., Leukemia 1996, 10, 1911-1918) is found in 20% of patients with acute myeloid leukemia (AML) while the most abundant mutation leading to a gain of function was found to be D835Y for which both compounds (1ath and 1bgi, FIG. 11) are more active than against the wild type (Y. Yamamoto, et al., Blood 2001, 97, 2434-2439). Other mutants (K663Q (M. Schittenhelm, et al., Leukemia 2006, 20, 2008-2014) and N841L (J. Jiang, et al., Blood 2004, 104, 1855-1858)) leading to a gain of function remained equally sensitive to the inhibitors. Likewise, the compound retained their activity against KIT mutants which results in a gain of function (D816V (C. Willmore-Payne, et al., Modern Pathology 2006, 19, 1164-1169) L576P (C. Willmore-Payne, et al., Modern Pathology 2006, 19, 1164-1169) and V559D (S. Hirota, et al., Science 1998, 279, 577-580)) however, these compounds are not active against the dual mutations which confers resistance to imatinib (V559D, T670I; V559D, V654A) (T. Carter, et al., Proc. Natl. Acad. Sci. USA 2005, 102, 11011-11016).

From a therapeutic perspective, the kinases inhibited by the cis-enone resorcylic acids contribute to the development, progression and aggressiveness of cancer. More recent in depth characterization of the specificity of kinase inhibitors have shown that all small molecule kinase inhibitors approved for therapeutic intervention or in clinical development do inhibit multiple targets (M. W. Karaman, et al., Nat. Biotechnol. 2008, 26, 127-132). The pallet of kinases inhibited by the cis-enone resorcylides all drive tumor growth. The fact that none of the compounds lacking the enone moiety fail to show significant activity testifies to the importance of the Michael acceptor as a selectivity filter. An important question is whether a mutation in the cysteine residue which is so important for the activity would be viable. However, based on the number of kinases inhibited along the MAP kinase cascade, a concerted evolution of several kinases would be necessary to evade the growth inhibition of these resorcylic acid lactones. The irreversible nature of the inhibition may prove to be important in achieving long lasting inactivation of specific pathways. The EGFR receptor has also been the target of irreversible inhibitors exploiting a cysteine residue. Results from phase I clinical trial have shown that EKB-569 is safe (C. Erlichman, et al., J. Clin. Oncol. 2006, 24, 2252-2260). From a chemical biology standpoint, compounds which form covalent bonds to their target are particularly useful as they can be used to label the targeted kinases for activity-based profiling and imaging. It is interesting to note how frequently evolution has resorted to irreversible inhibition in the selection of bioactive secondary metabolites (C. Drahl, B. F. Cravatt, E. J. Sorensen, Angew. Chem. Int. Ed. Engl. 2005, 44, 5788-5809). It should also be noted that irreversible inhibition of kinases leveraged on a conjugate addition is an endogenous mechanism as has been demonstrated for the inhibition of IKB by cyclopentenone prostaglandins (A. Rossi, et al., Nature 2000, 403, 103-108).

Two analogs bearing an oxygen in the macrocycle and a larger macrocyclic ring (1agh, 1aej) where evaluated for their efficacy in cell using a renal carcino line (RENCA) and in an orthopedic mice renal carcinoma. As shown in FIG. 12, the cis-enone inhibitor performed better than known inhibitor having an IC50 in the 100 to 500 nM. Evaluation of these analogs in mice showed a clear reduction in tumor growth at 20 mg/kg for aej and at 20 and 10 mg/kg for agh (FIG. 15).

In conclusion, this library offers important structural activity relationship in the cis-enone resorcylic acids. Two modifications were found to independently and synergistically improve the activity of this series of compounds while a modification which dramatically simplifies the synthetic accessibility of these compounds was established. The fact that several members of this family are potent inhibitors of multiple notorious oncogenic kinases and effective in cell as well as in whole animals should heighten their therapeutic interest.

### Example 2

Hypothemycin and related resorcylic acid lactones (RAL) bearing a cis-enone moiety have emerged as an alternative pharmacophore to heterocyclic motifs with a unique selectivity filter based on the irreversible reaction with a subset of the kinome bearing a suitably positioned cysteine residue. Two prototypical examples of “edited” RAL were evaluated for antitumoral, antimetastatic and antiangiogenic efficacy in an orthotopic murine renal cell carcinoma (RENCA) model. While both compounds were good inhibitors of VEGFRs in vitro, and inhibited tumor growth in vivo with comparable efficacy to sunitinib, an FDA-approved VEGFRs inhibitor, one compound did promote lung metastasis to a similar extent as sunitinib while the other strongly inhibited lung metastasis. This study attests to the potential of irreversible kinase inhibitors and molecular editing of natural pharmacophores and provides encouraging results to a clinically significant problem.

Material and Method

Cell Proliferation Assay.

Murine RENCA cells were originally obtained from a tumor that arose spontaneously in the kidney of BALB/c mice. Histologically, RENCA consists of granular cell type adenocarcinoma, which is pleomorphic with large nuclei. To obtain RENCA-LN, RENCA cells were transduced using a retrovirus encoding a luciferase-neomycin fusion protein and selected using 1 mg/ml G418. RENCA-LN cells were plated with 1.500 cells/150 μl/well in DMEM supplemented with 10% FCS in 96 well culture dishes. The following day the serial diluted test samples (from 1 mM to 0.1 μM in DMSO) were mixed with medium (1:25) and 50 μl of the mix was added to each well of the cultured cells (1:4) resulting in a final DMSO concentration of 1%. Cells were grown for 72 h at 10% CO2 and 37° C. After 72 h RENCA-LC cells were lysed in 100 μL luciferase assay buffer, and 10 μl of the lysate was assayed for luciferase activity. The mean value of the low controls of each 96 well cell culture plate represents the background which was subtracted from all other data points of the respective plate. Raw data were converted into percent proliferation relative to the high control, which were set to 100%. 1050 determination was done with GraphPad Prism 5.01 software with constrain of bottom to 0 and top to 100 using a nonlinear regression curve fit with variable hill slope. The equation is a four-parameter logistic equation. Experiments were run in triplicates.

Divergent Synthesis of Resorcylic Acid Lactones 3 and 5.

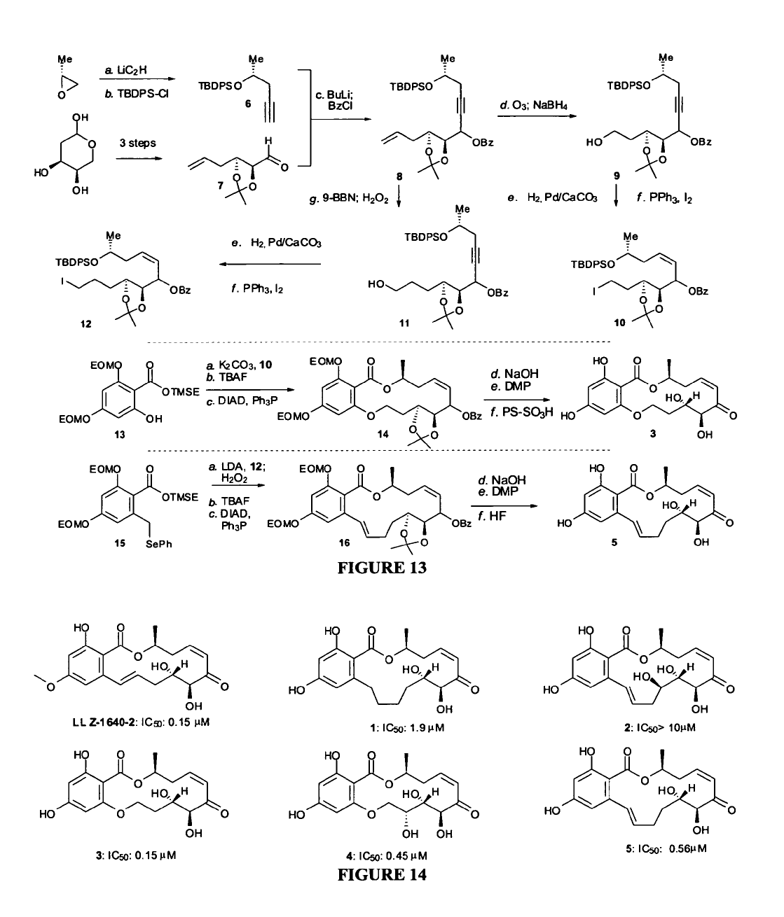

The gram-scale synthesis of compounds 3 and 5 is shown in FIG. 13. Lithium acetylide addition to propylene oxide followed by silylation of the crude reaction product afforded compound 6 which was deprotonated (nBuLi) and added onto aldehyde 7 readily obtained from D-2-deoxyribose in three steps (acetonide protection, (Barbat, J., et al. (1983) Carbohyd. Res. 116, 312-16) olefination (Geng, X. & Danishefsky, S. J. (2004) Org. Lett. 6, 413-6) and oxidation). Benzoate protection of the product afforded the key common intermediate 8 in good overall yield (x % from propelene oxide). Ozonolysis with a reductive workup afforded alcohol 9 wherein the alkene was reduced with Lindlar's catalyst and the alcohol converted to an iodide to obtain intermediate 10. Conversely, the terminal alkene of compound 8 was hydroborated with an oxidative work up to obtain alcohol 11 which was converted to idodie 12 after a Lindlar reduction of the alkyne. Following previously established chemistry, fragment 12 was coupled to the aromatic moiety 13 by standard alkylation of the phenol. Global silyl deprotection (TMSE and TBDPS) and Mitsunobu cyclization afforded 14. Selective hydrolysis of the benzoate, oxidation to reveal the cis-enone and global deprotection afforded macrocycle 3 in x % from 10. The larger macrocycle was prepared by alkylation of 12 with the aryl moiety 14 bearing a benzylic selenide by alkylation followed by oxidation/elimination of the selenide (Dakas, P. Y., et al. (2007) Angew. Chem. Int. Ed. Engl. 46, 6899-902). Using the same procedure as above with the exception of the final deprotection (HF rather than sulfonic acid resin), compound 5 was obtained in x % from 12 via 15.

In vivo efficacy

RENCA-LN cells were obtained and cultured as described above. Sixty female BALB/c mice of 5 to 6 weeks were orthotopically implanted RENCA-LN cells into the left kidney (4×10⁵ cells in 25 μl aliquots into the subcapsular space through a flank incision, Day 0). The mice were randomly divided into 5 groups of 12 mice each. On day 2, therapy was initiated. Animals in the vehicle control group (group 1) received 10 ml/kg Vehicle (PBS w/o Ca/Mg, 10% DMSO, 40% PEG-300) i.p. once daily until day 19. Animals of positive control group (group 2) received 40 mg/kg Sunitinib (Sutent®) p.o. once daily until day 20. Animals of treatment group 3 and 4 received 20 mg/kg of 3 once daily and every other day respectively (i.p.). Animals of treatment group 5 received 20 mg/kg of 5 once daily (i.p.). Due to partial weight loss, therapy in Groups 3 and 5 was suspended on days 11 and 12 and stopped on day 17. On day 20, the study was terminated and a necropsy performed. All animals were weighted, anaesthetised by isoflurane and killed by exsanguination. Two animals in group 1 were found dead on day 19 and 1 animal in group 3 and 5 on day 11 and 9 respectively. At necropsy, primary tumor weight and volume, lung weight and number of lung metastases were determined. Primary tumor tissues were collected and divided into two parts. One part of each tumor was snap-frozen in liquid nitrogen and stored at −80° C. whereas the other part was analyzed via ex vivo bioluminescence imaging. During the course of the study, the growth of the orthotopically implanted RENCA tumors was monitored on days 7, 14 and 19 using in vivo bioluminescence imaging. For this purpose, luciferin was injected intraperitoneally (i.p.) into the mice and light emission was measured post injection (10 min) using a NightOWL LB 981 bioluminescence imaging system (Berthold Technologies, Germany). The migration of RENCA tumor cells into the lung (metastasis) was analyzed post necropsy by counting nodules and using the luciferase assay. For this purpose, lungs were collected, homogenized, and assayed for luciferase activity using the kit from Promega (E1501) according to the manufacturer's instructions. Protein concentrations were determined using a Bradford assay and used to normalize luciferase activities (except for the primary tumor). A histological examination of the tumor vasculature was performed. For this purpose, cryosections of primary tumor tissues (thickness=5-10 μm) from the snap-frozen portion of the organ and blood vessels were visualized by immunohistochemical staining for CD 31 (PECAM-1; PharMingen, San Diego, Calif.). Statistical analysis: All relevant data of this study are graphically displayed as means±SEM. Statistical analysis of efficacy data was done using the Mann Whitney test.

Kinase Selectivity Profile

The selectivity profile was measure using the KinomeScan™ technology (http://www.kinomescan.com/) based on active-site dependent competition binding assay (Karaman, M. W., et al. (2008) Nat. Biotechnol. 26, 127-32; Fabian, M. A., et al. (2005) Nat. Biotechnol. 23, 329-36). Compounds 3 and 5 were profiled against a panel of 50 kinases including 42 kinases from the group of 48 kinases predicted by Santi et al. (Schirmer, A., et al. (2006) Proc. Natl. Acad. Sci. USA 103, 4234-9) to be targeted by hypothemycin and 5 mutants of FLT3 as well as 3 mutants of KIT. Both compounds were assayed at 1 μM. The results are shown in FIG. 10. The results are expressed as a percentage of signal between a negative (DMSO) and positive (know binder) control [(test compound−positive control)/(test compound−negative control)×100].

Results and Discussion

Multiple strategies for inhibiting the VEGF pathway have been shown to hinder tumor growth and the recent approval of small molecule inhibitors (sorafenib (Escudier, B., et al. (2007) N. Engl. J. Med. 356, 125-134; Abou-Alfa, G. K., et al. (2006) J. Clin. Oncol. 24, 4293-4300) and sunitinib (Motzer, R. J., et al. (2006) J. Clin. Oncol. 24, 16-24; Demetri, G. D., et al. (2006) Lancet 368, 1329-1338)) as well as neutralizing antibodies (bevacizumab (Miller, K., et al. (2007) N. Engl. J. Med. 357, 2666-2676; Hurwitz, H., et al. (2004) N. Engl. J. Med. 350, 2335-2342)) targeting the VEGF receptors have clinically validated this strategy (Folkman, J. (2007) Nat. Rev. Drug Discov. 6, 273-86). However, enduring clinical response are rare and there is mounting evidence that antiangiogenic therapy, while delaying primary tumor growth, can concomitantly promote tumor adaptation and progression to stages of greater malignancy with heighten invasiveness and distant metastasis (Bergers, G. & Hanahan, D. (2008) Nat. Rev. Cancer 8, 592-603; Kerbel, R. S. (2008) N. Engl. J. Med. 358, 2039-2049). It has recently been shown that short-term sunitinib treatment resulted in accelerated metastasis in an mice model (Ebos, J. M., et al. (2009) Cancer Cell 15, 232-9). Substantiating this pharmacological outcome, genetic ablation of Vegf-A increased invasiveness (Paez-Ribes, M., et al. (2009) Cancer Cell 15, 220-31). Furthermore, pretreatment of healthy mice with these VEGF inhibitors prior to inoculation of tumor cells “conditioned” the development of aggressive metastasis with shortened survival (Ebos, J. M., et al. (2009) Cancer Cell 15, 232-9). While several hypotheses have been advanced to rationalize these observations, the mechanism remains speculative (Loges, S., et al. (2009) Cancer Cell 15, 167-70). In most cancers, metastasis, not primary tumor growth, is ultimately fatal (Gupta, G. P. & Massague, J. (2006) Cell 127, 679-695). Yet, most preclinical studies focus on primary tumor growth with less attention on metastasis. While sunitinib and sorafenib are very effective inhibitors of the angiogenic pathways and primary tumor growth, we sought to evaluate resorcylic acids, as the irreversible inhibition of the target kinases may provide different outcome (Copeland, R. A., et al. (2006) Nat. Rev. Drug Discov. 5, 730-9; Swinney, D. C. (2004) Nat. Rev. Drug Discov. 3, 801-8; Potashman, M. H. & Duggan, M. E. (2009) J. Med. Chem.). Several programs to develop irreversible kinase inhibitors have already progressed to the clinic (Erlichman, C., et al. (2006) J. Clin. Oncol. 24, 2252-60; Janne, P. A., et al. (2007) J. Clin. Oncol. 25, 3936-44; Eskens, F. A., et al. (2008) Br. J. Cancer 98, 80-5).

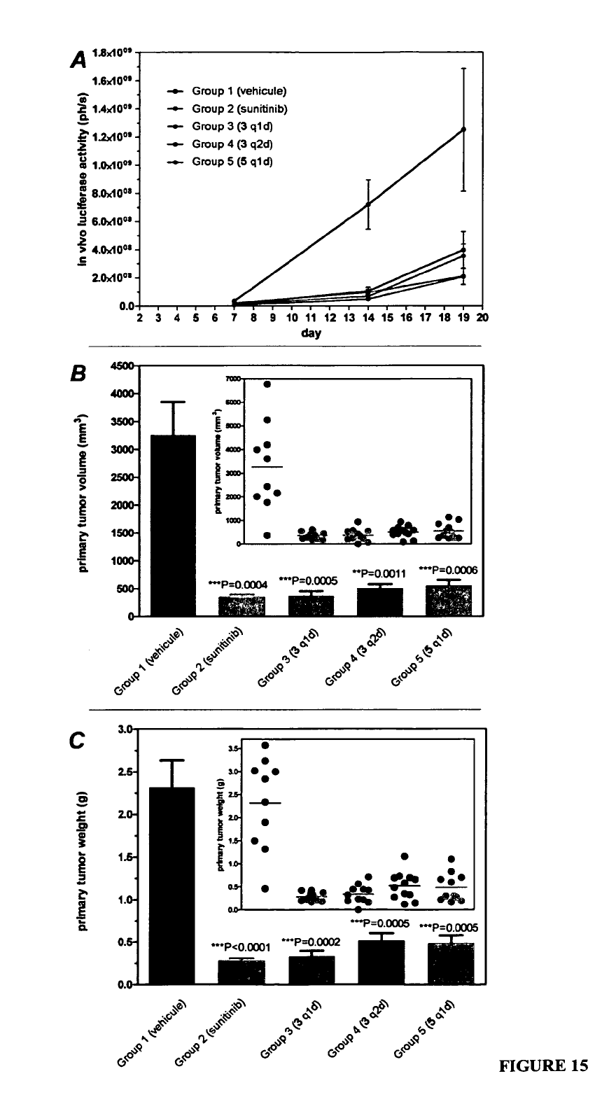

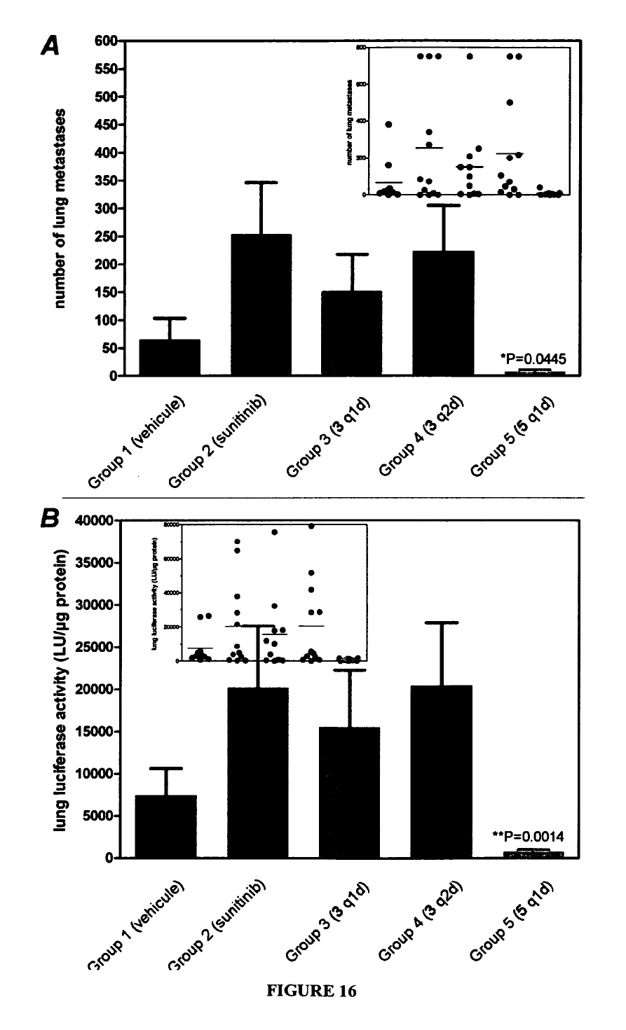

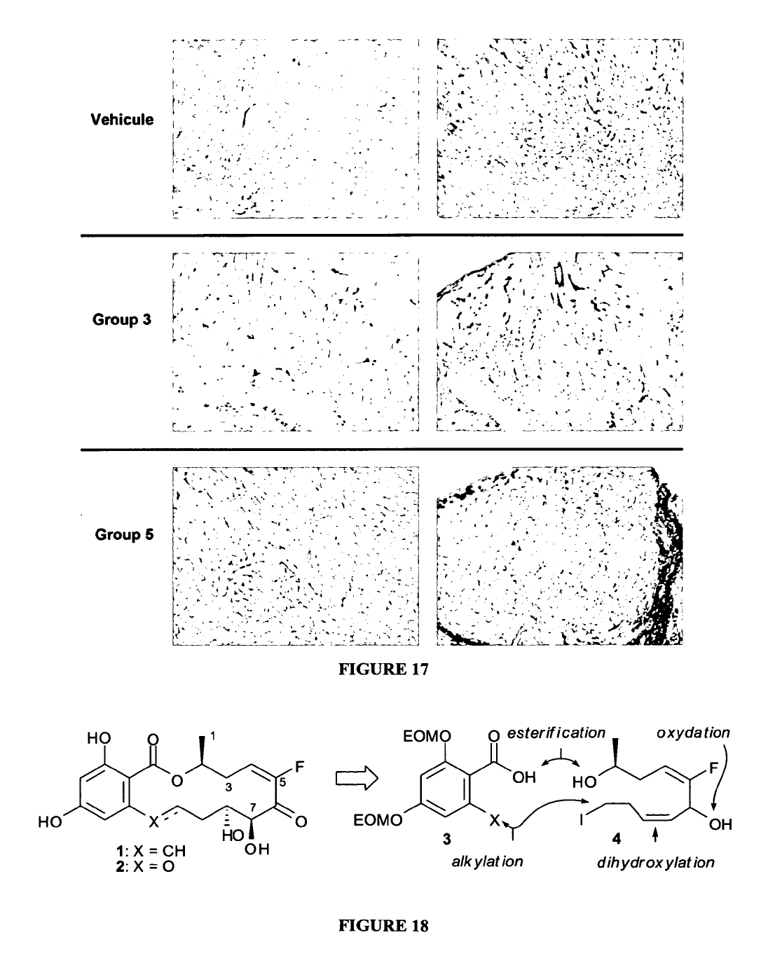

Following our biochemical screening, five cis-enone RAL analogues containing modifications which were found to be tolerated or beneficial in a biochemical assay were tested for their efficacy in inhibiting renal carcinoma cell line. As a bench mark, the natural product LL Z-1640-2 was also included in the assay. As shown in FIG. 14, compound 3 containing the benzylic ether was as effective as the natural product while the larger macrocycles were slightly less effective with the best (5) being nearly 3 fold less potent in growth inhibition. Compound 3 and 5 were selected as prototypical examples of modified RALs for further investigations. As described in the method section, both of these compounds derived from a common synthetic intermediate and can be readily prepared in gram quantities. While compound 3 was notably more soluble in aqueous buffers than 5, both compounds were easily formulated in 10% DMSO, 40% PEG, 60% saline. A first dose finding study indicated systemic toxicity for both compounds at high dose (75 mg/kg) but were tolerated at 25 mg/kg. Animal weights decreased between day 2 and day 5 (e.g. 8% decrease in case of group treated with 3 and 13% decrease for the group treated with 5 (q1d), but remained stable afterwards. Compound 3 was tested with two different schedules (every day dosing and every other day dosing) while compound 5 was tested at a single schedule (every day dosing) and the results were compared to a positive control with sunitinib. Both test compounds (3 and 5) showed a highly significant reduction of tumor growth as measure by bioluminescence (FIG. 15) and primary tumor volume with P values>0.0010 comparable to sunitinib (P=0.0004) for the daily dosing. Compound 3 administered every other day showed a marginal decrease activity (P=0.0011). It should be noted that we and others have found that cis-enone RAL such as LL Z-1640-2, compound 2 and 5 are inactivated by thiols such as DTT or GST and have short plasma half-life (Du, H., et al. (2009) Bioorg. Med. Chem. Lett. 19, 6196-9). Thus the marginal difference in activity between the two dose schedule of compound 3 suggests that the irreversible inhibition of its target is fast relative to compound inactivation and, that target turn over is slower than 12 h. Primary tumor weight confirmed these observations. A key objective of this study was to measure the effect of these compounds on metastasis. Upon termination of the study, the lungs were inspected for metastatic nodules and the ex-vivo luciferase activity of lung homogenates was measured. Concurring other studies and clinical observations, treatment with sunitinib resulted in a significant increase in lung metastastatic nodules with 5 animals having a high count of nodules whereas the untreated group had only two animals (FIG. 16). The overall level of luciferase activity from lung homogenates was three fold higher in the sunitinib treated group than in the untreated group. A similar result was obtained with compound 3 irrespective of the dosing schedule. However, within the group treated with compound 5, no animal was found with a high count of lung metastasis (P=0.0445) nor luciferase activity in lung homogenates (P=0.0014). Surprisingly, tumor vasulature histology (CD-31 staining) showed a strong inhibition of angiogenesis for animal treated with compound 3 but not for animals treated with compound 5 (FIG. 17). Comparison of the profile of kinase inhibition for both compound 3 and 5 showed a very similar pattern (FIG. 10) with VEGFRs being strongly inhibited in both cases. However, considering that both compounds 3 and 5 have a short plasma half life, difference in the kinetics of kinase inactivation in vivo relatively to inhibitor inactivation may become significant. It should be noted that these compounds were also shown to inhibit the inflammation pathways which have been implicated in facilitating metastasis. Finally, while hypothemycin had already been tested in mice bearing colon and lung tumors, only modest growth inhibition was observed (Tanaka, H., et al. (1999) Jap. J. Cancer Res. 90, 1139-1145).

In conclusion, the cis-enone resorcylic acid lactones have emerged as highly promising agent in oncology and worthy of further development. The molecular editing of this natural pharmacophore has revealed important differences in biological activity which are the product of subtle differences in biochemical activity and kinetics of inactivation. In most cases, metastasis, not primary tumor growths, kills the cancer patient. The inhibition of lung metastasis in the present study is thus highly interesting considering that current first line therapy based on angiogenesis promotes it.

Synthesis of Compounds

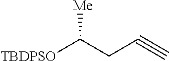

TBDPS protected chiral alkyne (#): To a stirred suspension of lithium acetylide.EDA complex (1.3 equiv, 37.6 g, 408.7 mmol) in DMSO (330 mL) was added epoxide (1.0 equiv, 18.2 g, 314.4 mmol) at 0° C. and the resulting mixture was stirred for 1 h at 0° C. and 14 h at 23° C. the reaction mixture was poured on ice (200 g), stirred for 30 min, extracted with CH₂Cl₂ (3×200 mL), the combined organic layers were washed with brine (200 mL), dried over Na₂SO₄, filtered and the crude solution was submitted for next step with out further evaporation of the solvents (R_{f}=0.25 (PE/EtOAc 4:1). To the above solution (660 mL) was added sequentially imidazol (2.0 equiv, 42.8 g, 628.8 mmol) and TBDPSC1 (1.1 equiv, 89.9 mL, 345.8 mmol) at 23° C. The resulting mixture was stirred for 18 h at the same temp before adding another portion of imidazol (2.0 equiv, 42.8 g, 628.8 mmol) and TBDPSC1 (0.5 equiv, 40.8 mL, 157.2 mmol). After stirring the reaction mixture for another 30 h, it was diluted with H₂O (200 mL), the layers were separated and the aqueous layer was extracted with CH₂Cl₂ (3×100 mL). The combined organic layers were washed with brine (300 mL), dried over Na₂SO₄, filtered and the solvents were evaporated under reduced pressure. The residue was purified by flash chromatography (SiO₂, PE, PE/EtOAc 33:1; PE/EtOAc 20:1) to furnish TBDPS protected chiral alkyne # (68.8 g, 68%) as a colorless oil.

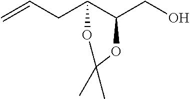

Alcohol (#): NaHDMS (2.8 equiv, 241.2 mL, 241.2 mmol, 1M in THF) was added to a suspension of methyltriphenylphosphoniumbromide (3.0 equiv, 92.2 g, 258.3 mmol) in THF (375 mL) at −78° C. The mixture was stirred at 23° C. for 30 min before the addition of sugar # (1.0 equiv, 15.0 g, 86.1 mmol) in THF (50 mL) at −78° C. Later the reaction mixture was stirred at 23° C. for 1 h, quenched with sat.aq.NH₄Cl (100 mL) before the reaction mixture turns red from yellow solution. The layers were separated, aqueous layer was extracted with EtOAc (3×100 mL) and the combined organic layers were dried over Na₂SO₄, filtered and evaporated under reduced pressure. The residue was purified by flash chromatography (SiO₂, PE/EtOAc 5:1) to obtain alcohol # (10.7 g, 72%) as a colorless liquid.

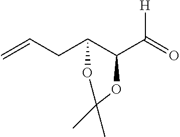

Aldehyde (#): To a solution of alcohol # (1.0 equiv, 10.0 g, 58.6 mmol) in CH₂Cl₂ (450 mL) and DMSO (115 mL) was added sequentially Et₃N (4.9 equiv, 39.5 mL, 284.5 mmol) and SO₃-pyridine complex (3.47 equiv, 32.0 g, 201.5 mmol) at 0° C. The reaction mixture was stirred for 20 min at the same temperature and 1 h at 23° C. before it was quenched with H₂O (200 mL). The layers were separated and the aqueous layer was extracted with CH₂Cl₂ (2×100 mL), the combined organic layers were washed with H₂O (200 mL), brine (200 mL), dried over Na₂SO₄, filtered and the solvents were evaporated under reduced pressure. The residue was purified by flash chromatography (SiO₂, PE/EtOAc 4:1) to provide aldehyde # (8.5 g, 86%) as a light yellow liquid.

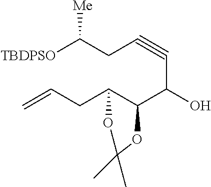

Allylic alcohol (#): To a solution of alkyne # (1.1 equiv, 17.7 g, 55.0 mmol) in THF (128 mL) was added n-BuLi (1.15 equiv, 42.6 mL, 57.5 mmol, 1.35 M in hexane) at −78° C. The reaction mixture was stirred for 45 min at the same temperature before aldehyde (1.0 equiv, 8.5 g, 50.0 mmol) in THF (128 mL) was added. After being stirred for 1.5 h at −78° C., 2 h at 0° C. and 30 min at 23° C., the reaction was quenched with Sat. aq. NH₄Cl (100 mL), warmed to RT, the layers were separated and the aqueous layer was extracted with EtOAc (3×50 mL), the combined organic layers were washed with brine (100 mL), dried over Na₂SO₄, filtered and concentrated at vacuo. The crude product was purified by flash chromatography (SiO₂, PE/EtOAc 20:1, 5:1, 4:1) to obtain alcohol # (19.8 g, 81%) as light yellow oil.

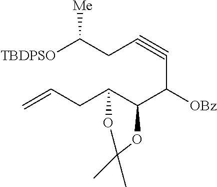

Benzoate (#):To a solution of alcohol # (1.0 equiv, 19.2 g, 38.9 mmol) in CH₂Cl₂ (230 ml) was added sequentially pyridine (2.5 equiv, 7.87 mL, 97.4 mmol) and benzoyl chloride (2.5 equiv, 11.3 mL, 97.4 mmol) at 0° C. The mixture was stirred for 12 h by warming slowly from 0° C. to RT. Then, the reaction mixture was diluted with H₂O (100 mL), stirred for 30 min, layers were separated and the aqueous layer was extracted with CH₂Cl₂ (3×50 mL). The combined organic layers were washed with sat. aq. NH₄Cl (100 mL), brine (100 mL), dried over Na₂SO₄, filtered and the solvent was removed in vacuo. The residue was purified by flash chromatography (PE, PE/EtOAc 50:1, 25:1) to obtain benzoate # (19.8 g, 85%) as a colorless oil.

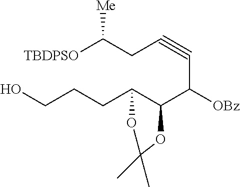

Alcohol (#): To a solution of alkene # (1.0 equiv, 21.7 g, 36.4 mmol) in THF (380 mL) was added 9-BBN (2.5 equiv, 182 mL, 91.0 mmol, 0.5M in THF) at 0° C. The reaction mixture was warmed to 23° C. and stirred for 14 h hours. After the flask was cooled to 0° C. 3N aq. NaOH solution (36.4 mL) followed by 30% aq. H₂O₂ (36.4 mL) was added very slowly, then the mixture was warmed to 23° C. and stirred for another 1.5 hours. The reaction mixture was quenched with H₂O (200 mL), the layers were separated, the aqueous layer was extracted with EtOAc (3×100 mL) and the combined organic layers were washed with brine (200 mL), dried over Na₂SO₄, filtered and evaporated under reduced pressure. The residue was purified by flash chromatography (SiO₂, PE/EtOAc 3:1, 2:1, 1:1) to provide the alcohol # (21.3 g, 95%) as a colorless liquid.

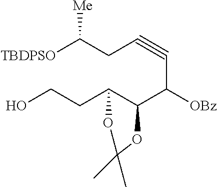

Aldehyde (#): A solution of alkene (1.0 equiv, 8.0 g, 13.4 mmol) in CH₂Cl₂/MeOH (130 mL) was bubbled with ozone for 30 min at −78° C. The excess ozone was removed by bubbling oxygen (the oxygen was bubbled until the blue colour disappeared). Later the mixture was bubbled using N₂ for 15 min before NaBH₄ (5 equiv, 2.53 g, 67.0 mmol)) was added. The reaction mixture was stirred for 30 min and added another portion of NaBH₄ (5 equiv, 2.53 g, 67.0 mmol), stirred the resulting mixture for 1 h at the same temp, placed the flask at 0° C., stirred for another 30 min before it was quenched with H₂O (50 mL), the reaction mixture was warmed to RT, extracted with EtOAc (5×50 mL), the combined organic layers were washed with brine (100 mL), dried over Na₂SO₄, filtered and the solvents were evaporated under reduced pressure. The residue was purified by flash chromatography (SiO₂, PE/EtOAc 4:1, 3:1, 2:1) to furnish alcohol # (4.0 g, 50%) as a colorless oil.

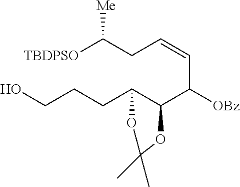

Alcohol #: To a solution of alcohol # (1.0 equiv, 21.2 g, 34.4 mmol) in MeOH (2.6 L) was added Pd/CaCO₃ (1.72 equiv, 6.3 g, 59.2 mmol) at 23° C. and the reaction mixture was stirred for 25 min under H₂ atmosphere (reaction was monitored by LC-MS). After filtration on celite, the solvents were evaporated under reduced pressure. The crude product was flushed from short pad of silica (PE/EtOAc 2:1) to obtain compound # (21.1 g, 99%) as a colorless oil.

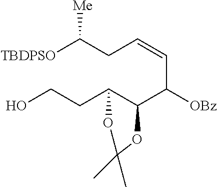

Compound #: The procedure of alcohol # was used with alcohol # (1 equiv, 4.41 g, 7.35 mmol) in MeOH (500 mL) and Pd/CaCO₃ (1.72 equiv, 1.34 g, 12.6 mmol) to yield compound # (4.30 g, 97%) as a colorless oil after filtration on short pad of silica.

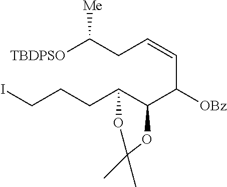

Alkyl iodide #: To a solution of alcohol # (1.0 equiv, 21.1 g, 34.2 mmol) in CH₂Cl₂ (125 mL) was added sequentially imidazole (2.0 equiv, 4.65 g, 68.4 mmol) and triphenylphosphine (1.8 equiv, 16.1 g, 61.6 mmol) at 23° C. The resulting mixture was cooled to 0° C., I₂ (1.8 equiv, 15.6 g, 61.6 mmol) was added and stirred at the same temperature for 10 min. After stirring the reaction mixture for 2.5 h at 23° C., it was quenched with sat. aq. NaHCO₃ (50 mL), the layers were separated and the aqueous layer was extracted with CH₂Cl₂ (3×25 mL). The combined organic layers were washed with sat. aq. Na₂S₂O₃ solution (50 mL), brine (50 mL), dried over Na₂SO₄, filtered and the solvents were evaporated under reduced pressure. The residue was purified by flash chromatography (PE/EtOAc 10:1) to afford the alkyl iodide # (22.3 g, 90%) as a light yellow oil.

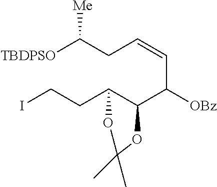

Iodide #: The procedure of iodide # was used with alcohol (1 equiv, 4.30 g, 7.13 mmol) in CH₂Cl₂ (30 mL), imidazole (3.0 equiv, 1.45 g, 21.4 mmol), triphenylphosphine (1.8 equiv, 3.36 g, 12.83 mmol) and I₂ (1.8 equiv, 3.25 g, 12.83 mmol) to yield iodide # (4.57 g, 90%) as a colorless oil after flash chromatography.

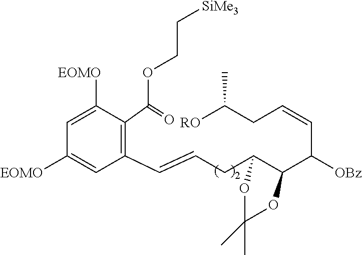

Procedure for alkylation reaction followed by the oxidation syn-elimination #: To a solution of aromatic seleno-ether (1.0 equiv, 16.3 g, 30.3 mmol) in THF/HMPA 10/1 (200 mL) cooled at −78° C. was added a freshly prepared solution of LDA in THF (2.0 equiv, 116.5 mL, 60.6 mmol, 0.52 M in THF) via cannula. The resulted dark red color reaction mixture was stirred for 10 min at −78° C. and a solution of the precooled alkyl iodide # (1.0 equiv, 22.0 g, 30.3 mmol) in THF (60 mL) was added dropwise at the same temperature. After stirring for 20 min, the reaction was quenched with sat. aq. NH₄Cl (100 mL), warmed the flask to room temperature, the layers were separated and the aqueous layer was extracted with EtOAc (3×50 mL). The combined organic layers were washed with brine (100 mL), dried over Na₂SO₄, filtered and the solvents were removed under reduced pressure. The residue was purified by flash chromatography (SiO₂, PE/EtOAc, 20:1, 10:1) to obtain desired alkylated compound # (30.9 g, 89%) as mixture of 4 diastereoisomers. R_{f}=0.26 (PE/EtOAc 6:1). To a stirred solution of previously prepared seleno-ether # (1.0 equiv, 30.9 g, 27.1 mmol) in THF (230 mL) was added H₂O₂ 35% (2.0 equiv, 6.5 mL, 61.8 mmol) at 23° C. After stirring for 13 h, the reaction mixture was concentrated under reduced pressure and the crude product was purified by flash chromatography (SiO₂, PE/EtOAc, 10:1) to afford compound # (24.2 g, 91%) as yellow oil.

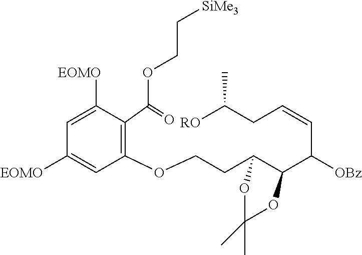

Compound #: To a solution of phenol # (1.0 equiv, 2.24 g, 5.81 mmol) in DMF (15 mL) was added K₂CO₃ (2.0 equiv, 1.55 g, 11.61 mmol) followed by a solution of iodide (1.1 equiv, 4.56 g, 6.39 mmol) in DMF (15 mL) at 23° C. The resulting mixture was heated to 80° C. and stirred for 16 h. then it was diluted with H₂O (25 mL), stirred for 20 min, extracted with Et₂O (4×25 mL), the combined organic layers were washed with brine (25 mL), dried over Na₂SO₄, filtered and the solvents were evaporated under reduced pressure. The reside was purified by flash chromatography (PE/EtOAc 10:1, 5:1) to afford the compound # (5.42 g, 96%) as a very light yellow oil.

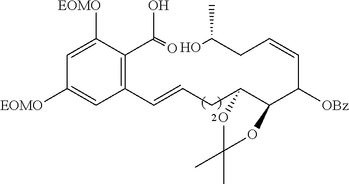

Acid #: To a solution of silyl-ester (1.0 equiv, 11.2 g, 11.4 mmol) in THF (155 mL) 23° C. was added tetra-butyl-ammonium fluoride (10.0 equiv, 114 mL, 114.1 mmol, 1M in THF). The resulting mixture was stirred for 56 h, then the reaction was quenched using sat. aq. NH₄Cl (100 mL), layers were separated, the aqueous layer extracted with EtOAc (5×50 mL), the combined organic layers were washed with H₂O (2×100 mL), dried over Na₂SO₄, filtered and concentrated under reduce pressure. The resulted crude product was subjected for the next step with out any further purification. LCMS: m/z for [M+Na]⁺ calcd for C₃₅H₄₆O₁₁Na: 665.293. found: 665.300.

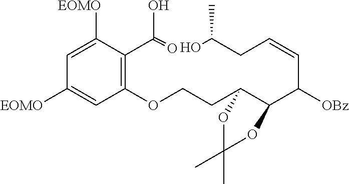

Acid #: The procedure of acid # was used with silyl ester # (1 equiv, 5.40 g, 5.56 mmol) in THF (80 mL), TBAF (10 equiv, 55.6 mL, 55.6 mmol, 1M in THF) to obtain acid # as a crude residue. LCMS: m/z for [M+Na]⁺ calcd for C₃₃H₄₄O₁₂Na: 655.273. found: 655.440.

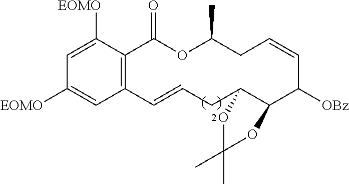

Macrocycle #: To a solution of acid # (1.0 equiv, 5.4 g, 8.4 mmol) in toluene (800 mL) at 23° C. was added DIAD (2.0 equiv, 3.32 mL, 16.9 mmol) and PPh₃ (2.0 equiv, 4.43 g, 16.9 Mmol) and the resulting mixture was stirred for 2 h. The solvents were removed under reduced pressure and the residue was purified by flash chromatography (SiO₂, PE/EtOAc, 10:1, 6:1) to obtain Mitsunobu product # (3.67 g, 70%) as a colorless oil.

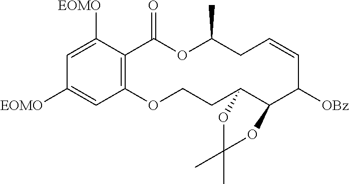

Macrocycle #: The procedure of macrocycle # was used with acid # (1 equiv, 2.58 g, 4.07 mmol) in toluene (380 mL), PPh₃ (2.0 equiv, 2.13 g, 8.15 mmol), DIAD (2.0 equiv, 1.60 mL, 8.15 mmol) to obtain Mitsunobu product # (2.05 g, 80%) as a colorless oil after flash chromatography.

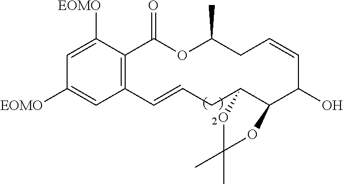

Allylic alcohol #: A solution of benzoate # (1.0 equiv, 3.67 g, 5.87 mmol) in 1% NaOH in MeOH (75 mL) was heated to 50° C. and stirred for 4 h. The solvents were removed under reduced pressure and the residue was purified by flash chromatography (SiO₂, PE/EtOAc, 6:1) to obtain allylic alcohol # (1.95 g, 64%) as colorless oil.

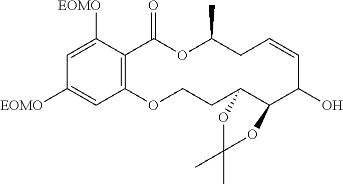

Allylic alcohol #: The procedure of macrocycle # was used with benzoate # (1 equiv, 2.0 g, 3.20 mmol) in 1% NaOH in MeOH (40 mL) to yield allylic alcohol # (1.13 g, 68%) as a colorless oil after flash chromatography.

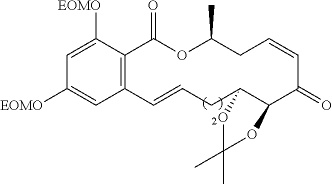

Cis-enone #: To a solution of allylic alcohol # (1.0 equiv, 1.90 g, 3.65 mmol) in CH₂Cl₂ (75 mL) was added DMP (1.5 equiv, 2.32 g, 5.47 mmol) at 23° C. The resulting mixture was stirred for 14 h, diluted with Et₂O (50 mL), quenched the reaction with 10% aq.

Na₂S₂O₃ (20 mL). After stirring the reaction mixture for 10 min, the layers were separated and the aqueous layer was extracted with Et₂O (3×10 mL), the combined organic layers were washed with sat. aq. NaHCO₃ (25 mL) and brine (25 mL), dried over Na₂SO₄, filtered and the solvents were evaporated under reduced pressure. The residue was filtered through short pad of silica (SiO₂, PE/EtOAc, 2:1) to obtain cis-enone # (1.62 g, 86%) as a light yellow oil.

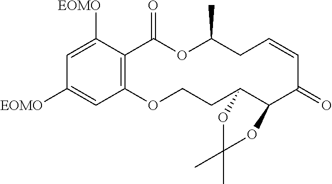

Cis enone #: The procedure of cis-enone # was used with alcohol # (1 equiv, 1.12 g, 2.15 mmol) in CH₂Cl₂ (45 mL) and DMP (1.5 equiv, 1.36 g, 3.22 mmol) to yield cis-enone # (910 mg, 83%) as a very light yellow oil.

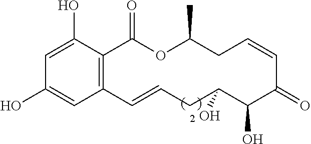

Macrocycle #: The cis-enone # (1.0 equiv, 1.0 g, 1.93 mmol) was diluted with HF 40% in H₂O/MeCN (1/10, 100 mL) at 23° C. The solution was stirred at 23° C. for 6.5 h, diluted with H₂O (100 mL), and then dried by lyophilization. The residue was purified by flash chromatography (SiO₂ was washed at least for 5 times with eluent before loading the compound, CH₂Cl₂/MeOH, 25:1) to afford the desired macrocycle # (490 mg, 70%) along with the corresponding para-mono-EOM compound that were resubmitted for a second deprotection in the same conditions. ¹H-NMR (CDCl₃, 400 MHz): δ=6.97 (d, J=15.9 Hz, 1H), 6.47 (dd, J=11.7, 3.1 Hz, 1H), 6.44 (d, J=2.4 Hz, 1H), 6.28 (d, J=2.4 Hz, 1H), 6.22-6.08 (m, 2H), 5.53-5.48 (m, 1H), 4.63 (d, J=2.4 Hz, 1H), 4.15 (dt, J=9.7, 3.0 Hz, 1H), 3.14 (dt, J=17.7, 10.9 Hz, 1H), 2.75 (dq, J=17.7, 2.7 Hz, 1H), 2.47-2.34 (m, 1H), 2.31-2.16 (m, 1H), 1.90-1.82 (m, 1H), 1.48 (d, J=6.2 Hz, 3H), 1.32-1.25 (m, 1H) ppm, 4OH signals not visible. ¹³C NMR (CDCl₃, 100 MHz, 25° C.): δ=202.2 (C═O), 172.9 (C═O), 166.7 (C), 164.0 (C), 146.1 (CH), 145.2 (C), 132.7 (CH), 132.5 (CH), 127.5 (CH), 109.4 (CH), 103.4 (C), 102.8 (CH), 82.1 (CH), 74.1 (CH), 69.6 (CH), 38.3 (CH₂), 30.0 (CH₂), 26.7 (CH₂), 20.9 (CH₃); HRMS (MALDI-TOF) m/z [M+H]⁺ calcd for C₁₉H₂₂O₇H, 363.1444. found: 363.1443.

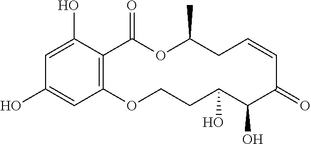 [SMILES: C[C@H]1C/C=C\C(=O)[C@@H](O)[C@@H](O)CCOc2cc(O)cc(O)c2C(=O)O1]

Oxygen Macrocycle #: To a solution of cis-enone # (1.0 equiv, 1.05 g, 2.06 mmol) in MeOH (185 mL) was added sulfonic acid resin (5.0 equiv, 3.33 g, 10.3 mmol, 3.0 mmol/g) at 23° C. The reaction mixture was shaken for 9 h, then it was filtered washed with CH₂Cl₂ (5×50 mL) and the solvents were removed under reduced pressure. The residue was precipitated by Et₂O to obtain desired macrocycle # (435 mg, 60%) as a very light red solid. R_{f}=0.34 (CH₂Cl₂/MeOH, 9:1); 10% trans isomer was observed in ¹H-NMR (CDCl₃, 400 MHz): δ=6.56 (d, J=15.6 Hz, 0.1H), 6.50 (dd, J=11.7, 3.0 Hz, 0.9H), 6.21 (td, J=11.6, 2.4 Hz, 1H), 6.00 (d, J=2.1 Hz, 1H), 5.97 (d, J=2.1 Hz, 1H), 5.43-5.36 (m, 1H), 4.67 (bs, 1H), 4.27-4.17 (m, 2H), 3.91 (dt, J=9.1, 3.0 Hz, 1H), 3.21 (dt, J=16.4, 11.6 Hz, 1H), 2.53-2.42 (m, 1H), 1.87 (ddd, J=14.5, 10.5, 3.5 Hz, 1H), 1.67-1.55 (m, 1H), 1.45 (d, J=6.2 Hz, 0.3H), 1.38 (d, J=6.2 Hz, 2.7H) ppm, 4OH signals not visible. ¹³C NMR (CDCl₃, 100 MHz, 25° C.): δ=202.3 (C═O), 172.6 (C═O), 167.1 (C), 165.4 (C), 163.7 (C), 145.7 (CH), 128.3 (CH), 96.5 (CH), 96.4 (C), 92.7 (CH), 82.9 (CH), 73.4 (CH), 69.9 (CH), 65.7 (CH₂), 37.8 (CH₂), 31.4 (CH₂), 20.9 (CH₃); HRMS (MALDI-TOF) m/z [M+H]⁺ calcd for C₁₇H₂₀O₈H, 353.1237. found: 353.1243.

### Example 3

Based on the fact that these compounds react with the cysteine residue through a Michael addition, modulation of the electronic properties of the enone may further enhance the inhibitory properties of this pharmacophore. We had shown that substitution at the β-position of the enone with a methyl group abrogated activity (Jogireddy, R., et al. (2009) Chem. Eur. J. 15, 11498-506). These results were concurrently confirmed by researchers from Eisai (Du, H., et al. (2009) Bioorg. Med. Chem. Lett. 19, 6196-9). To further pursue modulation of the Michael acceptor, we reasoned that a fluorine at the α-position of the enone may accentuate the Michael acceptor properties while constituting a fairly neutral steric permutation. This “molecular editing” (Wilson, R. M. & Danishefsky, S. J. (2006) J. Org. Chem. 71, 8329-51) may provide further benefits from a synthetic perspective as well as favorably impede the propensity of these cis-enones to isomerize to the thermodynamically more favorable and biologically less active trans-enone. As shown in FIG. 18, the synthesis could capitalize on our previously established strategy (Dakas, P. Y., et al. (2007) Angew. Chem. Int. Ed. Engl. 46, 6899-902; Dakas, P. Y., et al. (2009) Chem. Eur. J. 15, 11490-7; Barluenga, S., et al. (2006) Angew. Chem. Int. Ed. Engl. 45, 3951-3954), namely, disconnection of the ester and benzylic position, however, the difference of reactivity between a fluoroalkene and an alkene could be harnessed to introduce the diol moiety by dihydroxylation chemistry.

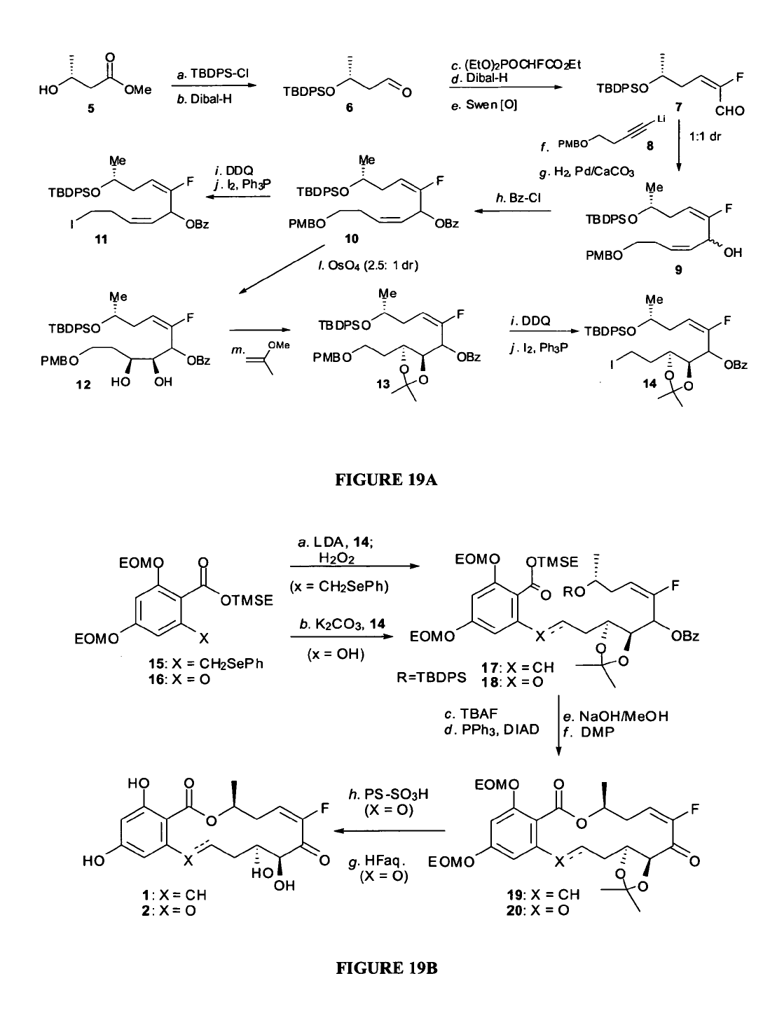

The synthesis commenced with the straightforward conversion of ester 5 into fluoroenal 7 via aldehyde 6 relying on a Horner-Wadsworth-Emmons reaction with triethyl 2-fluoro-2-phosphonoacetate to install the desired E-fluoroenoal (FIG. 19). Reaction of aldehyde 7 with alkyl lithium 8 afforded the desired alcohol as a 1:1 diasteromeric mixture which was reduced to the cis-alkene using Lindlar's catalyst (H₂, Pd/CaCO₃) to afford compound 9. As the hydroxyl group will ultimately be oxidized to the ketone, the lack of selectivity is inconsequential. Benzoyl protection of alcohol 9 thus afforded 10 wherein the terminal OPMB was converted to an iodide 11 (DDQ; I₂, PPh₃, imidazole). Attempts to engage this compound in alkylation chemistry with a phenol led to rapid elimination of the iodide to give a conjugated triene (product not shown). To circumvent this elimination which was thought to be facilitated by the presence of the alkene, compound 10 was dihydroxylated (2.5:1 dr in favor of the desired isomer 12) and converted to acetonide 13. Conversion of the terminal protected PMB ether into an iodide afforded the key intermediate 14. Following previously established chemistry, fragment 14 was coupled to the aromatic moiety 15 bearing a benzylic selenide by alkylation followed by oxidation/elimination of the selenide to afford 17. Conversely, fragment 14 was alkylated with phenol 16 to obtain the ether analogue 18 in excellent yield. Global deprotection of the silyl groups (TMSE and TBDPS) followed by Mitsunobu macrocyclization, selective benzoate deprotection and oxidation of the resulting alcohol afforded products 19 and 20 from 17 and 18 respectively in good overall yield. Global deprotection with aqueous HF or sulfonic acid resin afforded the desired fluoroenone resorcylic acids 1 and 2. We and others had previously noted the sensitivity of the cis-enone system of LL-Z1640-2 and related analogues to acid conditions which could lead to isomerisation (Dakas, P. Y., et al. (2009) Chem. Eur. J. 15, 11490-7; Hofmann, T. & Altmann, K.-H. (2008) Synlett, 1500-1504; Selles, P. & Lett, R. (2002) Tet. Lett. 43, 4627-4631). As anticipated based on the higher thermodynamic stability of E-fluoroalkene, analogues 1 and 2 proved to be more resistant to epimerization and no trace of isomerization was observed even with prolonged reaction times. Interestingly, the different diastereoisomers originating from the unselective dihydroxylation proved to have significant difference in the kinetics of deprotection which offered a convenient means to separate them.

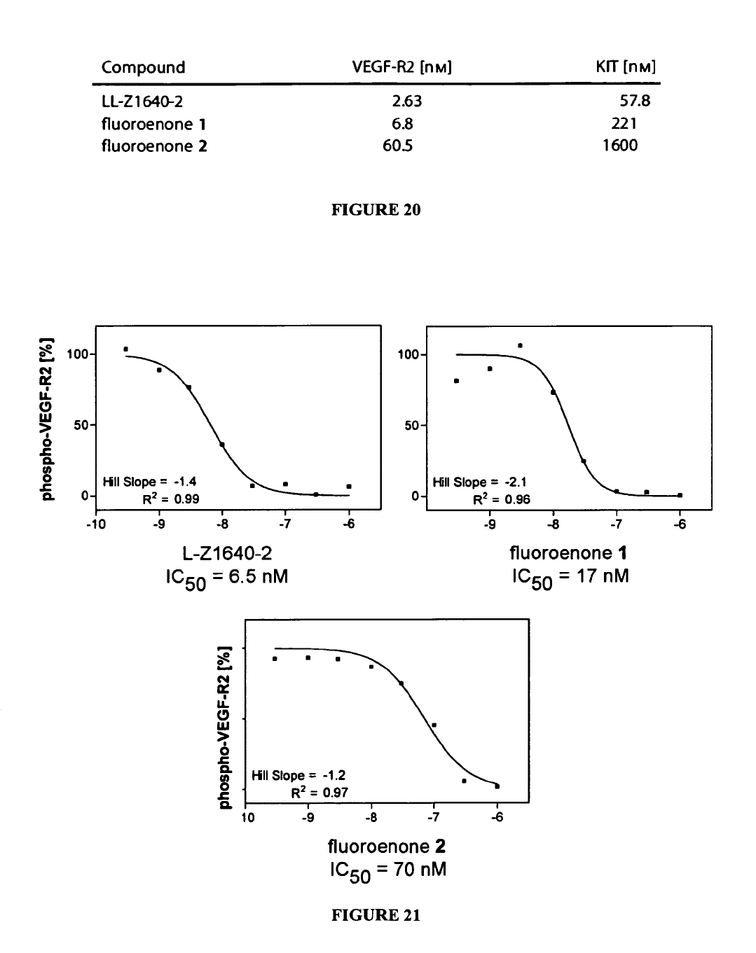

To gage the undiscriminate reactivity of these enone resorcylic acids with thiols, fluoroenone 2 and the parent analogue lacking the fluoride were treated with one equivalent of DTT (2 mM) as a prototypical thiol nucleophile in PBS (pH 7.4) containing 1% DMSO. Interestingly, a clear difference in reactivity was observed with the fluoroenone 2 reacting slower. With fluoroenone 1 and 2 in hand, we tested their efficacy against VEGF-R2 which was frequently found to be the most inhibited kinase in our profile of cis-enone resorcylic acid library (Dakas, P. Y., et al. (2007) Angew. Chem. Int. Ed. Engl. 46, 6899-902; Jogireddy, R., et al. (2009) Chem. Eur. J. 15, 11498-506) and KIT which represents a less inhibited kinase. As shown in FIG. 20, the fluoroenone 1 and 2 are less active than the natural product LL-Z1640-2 however maintain an inhibition level which is interesting (6.8 nM and 60.5 nM for 1 and 2 respectively against VEGF-R2 vs 2.63 nM for LL-Z1640-2) and their selectivity for VEGF-R2 relative to KIT remains unaffected. The products originating from the minor diastereoisomers of the dihydroxylation were significantly less active. Their cellular activity against VEGF-R2 did parallel the biochemical activity. As shown in FIG. 21, the natural product LL-Z1640-2 is a very potent inhibitor with an IC₅₀ of 6.5 nM. The direct comparison with the fluoroenone 2 revealed an IC₅₀ of 17 nM while the analogue containing the synthetically more accessible ether macrocycle remained very potent with an IC₅₀ of 70 nM. Thus the fluoroenone modification is well tolerated but does not enhance the cellular efficacy of VEGF-R2 inhibition. During the course of our investigation, a similar modification was reported by researchers from Esai (Du, H., et al. (2009) Bioorg. Med. Chem. Lett. 19, 6196-9) however, their medicinal chemistry efforts focused the inhibition of inflammation related pathways (MEKs and their down-stream regulatory effect on cytokines) (Du, H., et al. (2009) Bioorg. Med. Chem. Lett. 19, 6196-9; Goto, M., et al. (2009) J. Pharmacol. Exp. Ther. 331, 485-95). A selected analogue (E6201) indeed shows a >60 and >200 fold selectivity (Goto, M., et al. (2009) J. Pharmacol. Exp. Ther. 331, 485-95) for MEK1 relatively to VGF-R2 and PDGFRβ, which is a significantly different pattern relatively to the parent natural product (LL-Z1640-2) for which VEGF-R2 and PDGFRβ are most inhibited (Jogireddy, R., et al. (2009) Chem. Eur. J. 15, 11498-506). It was further shown that the addition of a methyl substituent at the δ position to the enone enhanced the metabolic stability with a tolerable loss of activity (10 fold) (Du, H., et al. (2009) Bioorg. Med. Chem. Lett. 19, 6196-9). It should be note however that several cis-enone had already shown to be effective in vivo. As the inhibition is irreversible, the relative rate of metabolic instability and irrevesible inhibition coupled to target turn over should be considered.

Synthesis of Compounds

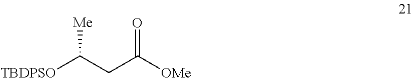

TBDPS protected chiral ester (21): To a stirred solution of alcohol 5 (1.0 equiv, 10.0 g, 84.6 mmol) in CH₂Cl₂ (150 mL) were added sequentially imidazol (2.0 equiv, 11.5 g, 169.2 mmol) and TBDPSCl (1.2 equiv, 26.5 mL, 101.5 mmol) at 0° C. The resulting mixture was stirred for 13 hours at 23° C. Then the mixture was diluted with ether (300 mL), washed with sat.aq.NH₄Cl (200 mL) and brine (200 mL), dried over Na₂SO₄, filtered and evaporated under reduced pressure. The residue was purified by flash chromatography (SiO₂, petroleum ether/EtOAc 9:1) to obtain 21 (27.2 g, 90%) as a colorless oil.

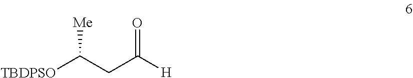

Aldehyde (6): To a solution of ester 21 (1.0 equiv, 10.0 g, 28.0 mmol) in toluene (150 mL) at −78° C. was added dropwise a 1M solution of DIBAL in toluene (1.1 equiv, 30.8 mL, 30.8 mmol) over a period of 30 min. The reaction mixture was stirred for 1 hour at −78° C. before it was quenched with MeOH (3.3 mL) and sat.aq.NH₄Cl (5.5 mL). The mixture was then diluted with Et₂O (200 mL), poured to a flask containing 50% aq. NaK tartarate (200 mL), stirred for 1 hour at room temperature, the layers were separated and the aqueous layer was washed with Et₂O (2×100 mL). The organic layers were combined and dried over Na₂SO₄, filtered and the solvents were evaporated under reduced pressure. The residue was purified by flash chromatography (SiO₂, petroleum ether/EtOAc 9:1) to afford aldehyde 6 (7.82 g, 85%) as a colorless oil.

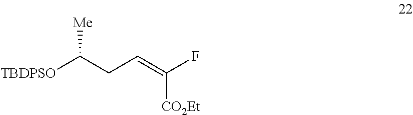

Allylic ester (22): To a solution of the phosphonate ester (1.2 equiv, 5.0 g, 20.6 mmol) in THF (200 mL) at 0° C. was added n-BuLi (1.2 equiv, 14.7 mL, 20.6 mmol, 1.4 M in hexane). The reaction mixture was stirred for 1 hour at the same temperature before the solution of aldehyde 6 (1.0 equiv, 5.60 g, 17.1 mmol) in THF (20 mL) was added. After 20 min the flask was placed at room temperature and stirred for 12 hours. The reaction was then quenched with 0.5 N aq. HCl (100 mL), the layers were separated and the aqueous layer was extracted with EtOAc (3×50 mL). The combined organic layers were washed with brine (100 mL), dried over Na₂SO₄, filtered and the solvents were removed under reduced pressure. The residue was purified by flash chromatography (SiO₂, petroleum ether/EtOAc 19:1) to afford ester 22 (5.92 g, 83%) as a light yellowish oil. It contains 6% Z-isomer.

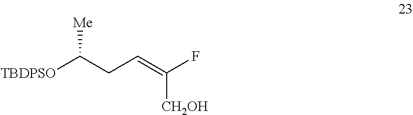

Allylic alcohol (23): To a solution of allylic ester 22 (1.0 equiv, 5.86 g, 14.1 mmol) in CH₂Cl₂ (160 mL) at 0° C. was added DIBAL (2.5 equiv, 35.3 mL, 35.3 mmol, 1M solution in toluene). The reaction mixture was stirred for 40 min at 0° C. and 30 min at room temperature, quenched with 50% aq. NaK tartarate solution (100 mL) and stirred for 2 hours more at the same temperature. The layers were separated and the aqueous layer was extracted with EtOAc (2×100 mL), the combined organic layers were washed with brine (200 mL), dried over Na₂SO₄, filtered and the solvents were evaporated under reduced pressure. The residue was purified by flash chromatography (SiO₂, petroleum ether/EtOAc 9:1) to afford allylic alcohol 23 (4.48 g, 85%) as a colorless oil.

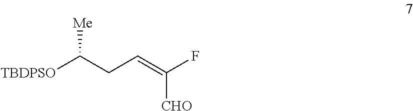

Aldehyde (7): To a solution of oxalyl chloride (1.5 equiv, 1.52 mL, 17.7 mmol) in CH₂Cl₂ (35 mL) at −78° C. was added DMSO (2.45 equiv, 2.2 mL, 28.9 mmol) in CH₂Cl₂ (7 mL). The mixture was stirred for 30 min at the same temperature before a solution of alcohol 23 (1.0 equiv, 4.4 g, 11.8 mmol) in CH₂Cl₂ (10 mL) was added. After stirring the reaction for 45 min at −78° C., Et₃N (4.0 equiv, 6.56 mL, 47.2 mmol) was added and the mixture was allowed to warmed to room temperature over a period of 2 hours. Then it was diluted with H₂O (25 mL), layers were separated and the aqueous layer was extracted with CH₂Cl₂ (2×20 mL), the combined organic layers were washed with 1N HCl (25 mL), sat. aq. NaHCO₃ (25 mL), and brine (25 mL), dried over Na₂SO₄, filtered and the solvents were evaporated under reduced pressure. The residue was purified by flash chromatography (SiO₂, petroleum ether/EtOAc 9:1) to furnish aldehyde 7 (4.30 g, 98%) as a colorless oil.

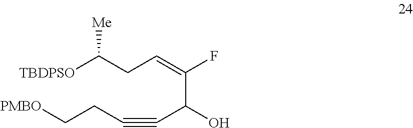

Alcohol 24: To a solution of the alkyne (2.06 equiv, 4.54 g, 23.9 mmol) in THF (227 mL) at −78° C. was added n-BuLi (2.0 equiv, 19.3 mL, 23.2 mmol, 1.2 M in hexane). The reaction mixture was stirred for 1 hour at the same temperature before adding aldehyde 23 (1.0 equiv, 4.30 g, 11.6 mmol) in THF (140 mL). After stirring the mixture for 1 hour at −78° C., the reaction was quenched with sat. aq. NH4Cl (100 mL) and warmed to 23° C. Once a room temperature the layers were separated and the aqueous layer was extracted with EtOAc (3×50 mL), the combined organic layers were washed with brine (100 mL), dried over Na₂SO₄, filtered and concentrated under vacuo. The crude product was purified by flash chromatography (SiO₂, petroleum ether/EtOAc 9:1, 4:1) to obtain alcohol 24 (4.05 g, 62%, 1:1 ratio of isomers) as a light yellow oil.

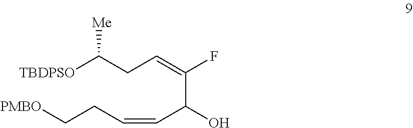

Compound 9: A solution of alcohol 24 (1.0 equiv, 3.39 g, 6.04 mmol) in MeOH (470 ml) at 23° C. was treated with Pd/CaCO₃ (1.72 equiv) and the reaction mixture was stirred for 25 min under H₂ atmosphere. After filtration on celite, the solvents were evaporated under reduced pressure. The crude product was flushed through a short pad of silica (petroleum ether/EtOAc 3:1) to obtain compound 9 (3.30 g, 97%) as a colorless oil.

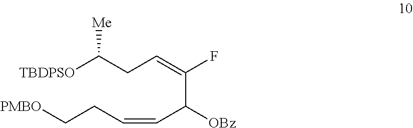

Benzoate 10: To a solution of alcohol 9 (1.0 equiv, 3.30 g, 5.86 mmol) in CH₂Cl₂ (35 ml) at 0° C. were added sequentially pyridine (2.5 equiv, 1.18 mL, 14.65 mmol) and benzoyl chloride (1.5 equiv, 1.02 mL, 8.79 mmol) and the mixture was stirred for 14 hours while warming slowly from 0° C. to room temperature. Then, the reaction was diluted with H₂O (20 mL), the layers were separated and the aqueous layer was extracted with CH₂Cl₂ (3×20 mL). The combined organic layers were washed with sat. aq. NH₄Cl (20 mL), brine (20 mL), dried over Na₂SO₄, filtered and the solvent was removed under vacuo. The crude product was purified by flash chromatography (petroleum ether/EtOAc 20:1, 15:1, 10:1) to obtain benzoate 10 (3.56 g, 91%) as a colorless oil.

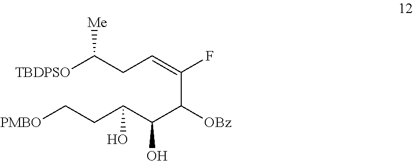

Diol 12: To a solution of olefin 10 (1.0 equiv, 702 mg, 1.05 mmol) in THF (7 mL) were added sequentially NMO.H₂O (2.0 equiv, 284 mg, 2.10 mmol) in H₂O (7 mL) and OsO₄ (0.013 equiv, 86 μL, 0.0136 mmol, 4% wt in H₂O) at 23° C. The resulted mixture was stirred for 15 hours. Then the reaction was quenched with a 1:1 mixture of sat. aq. NaHCO₃ and 10% aq. Na₂S₂O₃ solution (5 mL) and stirred at room temperature for 1 hour. The layers were separated and the aqueous layer was extracted with EtOAc (3×5 mL), the combined organic layers were washed with brine (10 mL), dried over Na₂SO₄, filtered and the solvents were evaporated under reduced pressure. The crude product was purified by flash chromatography (petroleum ether/EtOAc 6:1, 4:1, 3:1) to afford the diol 12 (577 mg, 78% based on isolated yield and 87% based on recovered starting material, 2.5:1 ratio of isomers from the dihydroxylation) as a colorless oil.

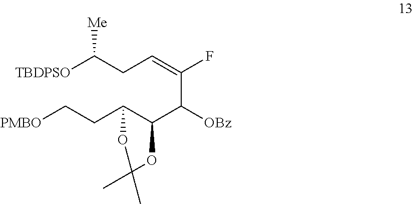

Acetal 13: To a solution of diol 12 (1.0 equiv, 577 mg, 0.823 mmol) in CH₂Cl₂ (10 mL) was added 2-methoxy propene (2.0 equiv, 0.15 mL, 1.64 mmol) followed by PPTS (0.1 equiv, 20.7 mg, 0.082 mmol) at 23° C. and the resulted solution was stirred 1 hour. Then the reaction was quenched with sat. aq. NaHCO₃ soln. (5 mL), stirred for 10 min, the layers were separated and the aqueous layer was extracted with CH₂Cl₂ (2×5 mL). The combined organic layers were washed with brine (10 mL), dried over Na₂SO₄, filtered and the solvents were evaporated under reduced pressure. The crude product was purified by flash chromatography (petroleum ether/EtOAc 4:1) to afford the acetal 13 (566 mg, 93%) as a colorless oil.

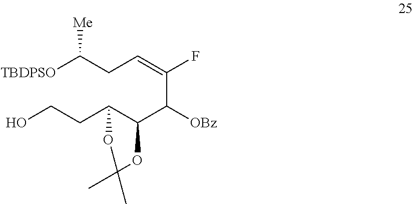

Alcohol 25: To a solution of PMB-ether 13 (1.0 equiv, 566 mg, 0.763 mmol) in CH₂Cl₂/H₂O 2/1 (35 mL) was added DDQ (1.2 equiv, 208 mg, 0.916 mmol) at 23° C. After stirring for 3 hours, the reaction mixture was diluted with Et₂O (10 mL), quenched with sat. aq. NaHCO₃ (10 mL) and stirred for 10 min. Then the layers were separated and the aqueous layer was extracted with Et₂O (3×5 mL). The combined organic layers were washed with H₂O (2×10 mL) and brine (10 mL), dried over Na₂SO₄, filtered and the solvents were removed under reduced pressure. The residue was purified by flash chromatography (SiO₂, petroleum ether/EtOAc, 9:1, 3:1) to obtain desired alcohol 25 (434 mg, 92%) as light yellow oil.

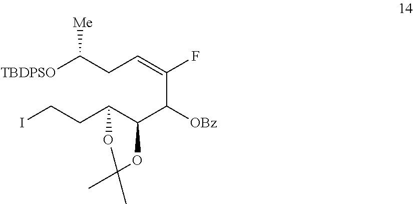

Alkyl iodide 14: To a solution of alcohol 25 (1.0 equiv, 434 mg, 0.699 mmol) in CH₂Cl₂ (5 mL) were added sequentially imidazole (3.0 equiv, 142.7 mg, 2.09 mmol), triphenylphosphine (1.8 equiv, 330 mg, 1.26 mmol) and I₂ (1.8 equiv, 319.2 mg, 1.26 mmol) at 23° C. and the resulted mixture was stirred for 1.5 hours. Then, the reaction was quenched with sat. aq. NaHCO₃ (5 mL), the layers were separated and the aqueous layer was extracted with CH₂Cl₂ (3×5 mL). The combined organic layers were washed with sat. aq. Na₂S₂O₃ solution (10 mL), brine (10 mL), dried over Na₂SO₄, filtered and the solvents were evaporated under reduced pressure. The residue was purified by flash chromatography (petroleum ether/EtOAc 9:1) to afford the alkyl iodide 14 (460 mg, 90%) as a light yellow oil.

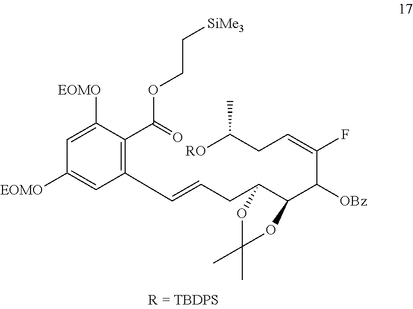

Synthesis of compound 17: To a solution of aromatic seleno-ether 15 (1.0 equiv, 334 mg, 0.62 mmol) in THF/HMPA 10/1 (5 mL) cooled at −78° C. was added a freshly prepared solution of LDA in THF (2.0 equiv, 2.58 mL, 1.24 mmol, 0.48 M in THF). The resulted dark red color mixture was stirred for 10 min at −78° C. and a solution of the precooled alkyl iodide 14 (1.0 equiv, 453 mg, 0.62 mmol) in THF (1 mL) was added dropwise at the same temperature. After stirring for 20 min, the reaction was quenched with sat. aq. NH₄Cl (5 mL), warmed the flask to room temperature, the layers were separated and the aqueous layer was extracted with EtOAc (3×5 mL). The combined organic layers were washed with brine (10 mL), dried over Na₂SO₄, filtered and the solvents were removed under reduced pressure. The residue was purified by flash chromatography (SiO₂, petroleum ether/EtOAc, 10:1) to obtain desired alkylated compound (569 mg, 80%) as mixture of diastereoisomers. R_{f}=0.28 (petroleum ether/EtOAc 5:1). To a stirred solution of previously prepared seleno-ether (1.0 equiv, 569 mg, 0.498 mmol) in THF (4.2 mL) was added H₂O₂ 35% (2.0 equiv, 0.12 mL, 0.996 mmol) at 23° C. After stirring for 3 hours, the reaction mixture was concentrated under reduced pressure and the crude product was purified by flash chromatography (SiO₂, petroleum ether/EtOAc, 5:1) to afford compound 17 (455 mg, 92%) as a yellow oil.

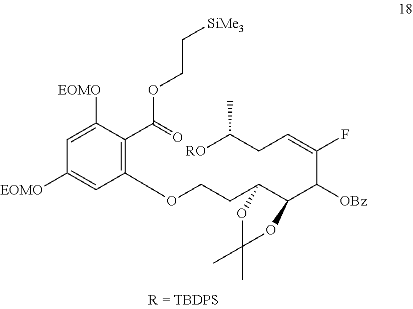

Compound 18: To a solution of phenol 16 (1.0 equiv, 192.3 mg, 0.497 mmol) in DMF (1.2 mL) was added K₂CO₃ (2.0 equiv, 137.3 mg, 0.994 mmol) followed by a solution of iodide (1.1 equiv, 400 mg, 0.547 mmol) in DMF (1.2 mL) at 23° C. The resulting mixture was heated to 80° C. and stirred for 12 hours. The reaction was then diluted with H₂O (5 mL), stirred for 20 min, extracted with Et₂O (5×5 mL), the combined organic layers were washed with brine (10 mL), dried over Na₂SO₄, filtered and the solvents were evaporated under reduced pressure. The residue was purified by flash chromatography (petroleum ether/EtOAc 9:1, 5:1) to afford compound 18 (490 mg, 99%) as a very light yellow oil.

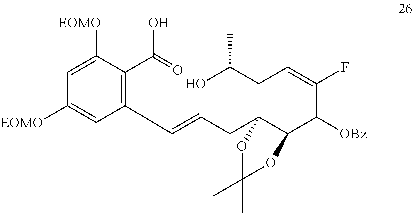

Acid 26: To a solution of silyl-ester 17 (1.0 equiv, 455 mg, 0.461 mmol) in THF (6.2 mL) was added tetra-butyl-ammonium fluoride (10.0 equiv, 4.6 mL, 4.6 mmol, 1M in THF) at 23° C. The resulting mixture was stirred for 44 hours. The reaction was then quenched using sat. aq. NH₄Cl (5 mL), the layers were separated, the aqueous layer extracted with EtOAc (5×5 mL), the combined organic layers were washed with H₂O (2×5 mL), dried over Na₂SO₄, filtered and concentrated under reduce pressure. The resulted crude product was subjected for the next step with out any further purification.

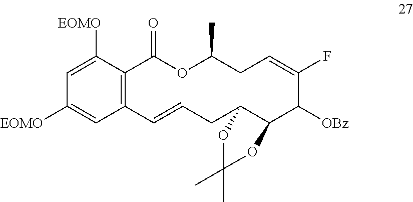

Macrocycle 27: To a solution of acid 26 in toluene (45 mL) were added DIAD (2.0 equiv, 0.18 mL, 0.922 mmol) and Ph₃P (2.0 equiv, 241.8 mg, 0.922 mmol) at 23° C. and the resulting mixture was stirred for 1.5 hours. Then, the solvents were removed under reduced pressure and the residue was purified by flash chromatography (SiO₂, petroleum ether/EtOAc, 10:1, 6:1) to obtain macrocycle 27 (216 mg, 74%) as a light yellow oil.

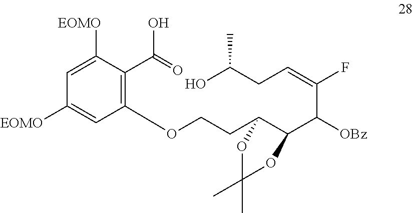

Following the same procedure as describe above silyl ester 18 (1.0 equiv, 490 mg, 0.495 mmol) in THF (6.7 mL), TBAF (15 equiv, 7.43 mL, 7.43 mmol, 1M in THF) was deprotected to obtain acid 28 that was submitted to the next step without any further purification.

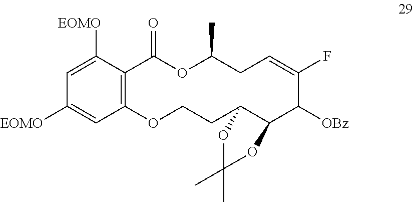

Following the same procedure as describe above acid 28 (1.0 equiv, 322 mg, 0.495 mmol) in toluene (47 mL) was treated with PPh₃ (2.0 equiv, 259.6 mg, 0.99 mmol) and DIAD (2.0 equiv, 0.19 mL, 0.99 mmol) to obtain, after flash chromatography, macrocycle 29 (220 mg, 70%) as a colorless oil.

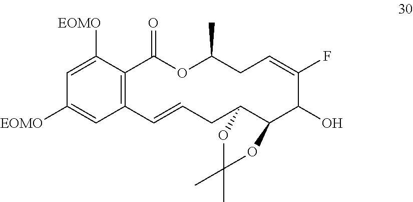

Allylic alcohol 30: A solution of benzoate 27 (1.0 equiv, 166 mg, 0.264 mmol) in 1% NaOH in MeOH (4 mL) was heated to 50° C. and stirred for 2 hours. Then, the solvents were removed under reduced pressure and the residue was purified by flash chromatography (SiO₂, petroleum ether/EtOAc, 5:1, 4:1, 2:1) to obtain allylic alcohol 30 (105 mg, 76%) as light yellow oil.

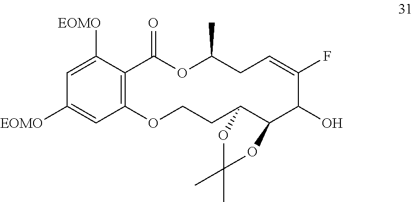

Following the same procedure as describe above alcohol 29 (1.0 equiv, 156 mg, 0.246 mmol) was treated with 1% NaOH in MeOH (3.2 mL) to yield allylic alcohols 31 (90 mg, 69%) as a colorless oil after flash chromatography.

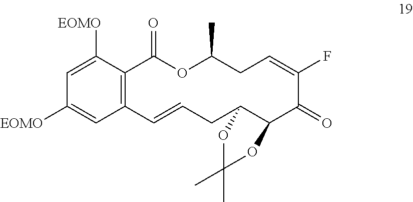

Cis-enone 19: To a solution of allylic alcohol 30 (1.0 equiv, 51 mg, 0.097 mmol) in CH₂Cl₂ (2 mL) was added DMP (1.5 equiv, 61.5 mg, 0.145 mmol) at 23° C. The resulting mixture was stirred for 12 hours, diluted with Et₂O (5 mL), and the reaction was finally quenched with 10% aq. Na₂S₂O₃ (2 mL). After stirring the mixture for 10 min, the layers were separated and the aqueous layer was extracted with Et₂O (3×1 mL), the combined organic layers were washed with sat. aq. NaHCO₃ (2 mL) and brine (2 mL), dried over Na₂SO₄, filtered and the solvents were evaporated under reduced pressure. The residue was purified by flash chromatography (SiO₂, petroleum ether/EtOAc, 5:1) to obtain cis-enone 19 (45 mg, 88%) as a light yellow oil.

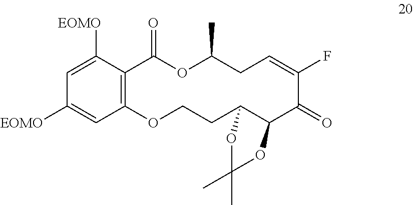

C is enone 20: Following the procedure described above compound 31 (1.0 equiv, 30 mg, 0.056 mmol) in CH₂Cl₂ (2 mL) was oxydized with DMP (1.5 equiv, 35.6 mg, 0.084 mmol) to yield cis-enone 20 (25 mg, 83%) as a very light yellow oil.

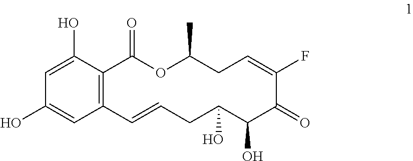 [SMILES: C[C@H]1C/C=C(/F)C(=O)[C@@H](O)[C@@H](O)C/C=C/c2cc(O)cc(O)c2C(=O)O1]

Macrocycle 1: Compound 19 (1.0 equiv, 40 mg, 0.076 mmol) was diluted with HF 40% in H₂O/MeCN (1/10, 4 mL) at 23° C. The solution was stirred for 6.5 hours, diluted with H₂O (4 mL), and then dried by lyophilization. The residue was purified by flash chromatography (SiO₂ was washed at least for 5 times with eluent prior to loading the compound, CH₂Cl₂/MeOH, 25:1) affording desired macrocycle 1 (14 mg, 50%) along with a mixture of compounds at different stages of deprotection. This mixture was submitted for a second deprotection reaction using this time PS—SO₃H resin/MeOH (10 equiv) at 50° C. After 7 hours the resin was filtered and was washed with CH₂Cl₂ (5×2 mL), the solvents were removed under reduced pressure and the residue was purified by HPLC [Supelco C18 (5 cm×10 mm, 5 uM) 16 to 20% acetonirtrile in water, 2 mL/min] to afford macrocycle 32 (5.5 mg, 20%) as a colorless compound as well as 4.5 mg (15%) of 1.

1: R_{f}=0.19 (CH₂Cl₂/MeOH 9:1); ¹H-NMR (MeOD, 400 MHz): δ=6.99 (d, J=15.3 Hz, 1H), 6.40 (d, J=2.4 Hz, 1H), 6.28 (d, J=2.4 Hz, 1H), 6.09 (ddd, J=15.1, 8.9, 6.4 Hz, 1H), 5.95 (ddd, J=21.9, 11.6, 2.7 Hz, 1H), 5.28-5.20 (m, 1H), 4.90 (bs, 1H), 4.09-4.04 (m, 1H), 3.50-3.39 (m, 1H), 2.62-2.53 (m, 1H), 2.31-2.17 (m, 2H), 1.50 (d, J=6.2 Hz, 3H) ppm, 4OH signals not visible; ¹³C NMR (MeOD, 100 MHz, 25° C.): δ=196.4 (d, ²J_{c,F}=36.8 Hz, C═O), 172.8 (C═O), 166.9 (C), 164.1 (C), 155.2 (2×d, ¹J_{c,F}=254.6 Hz, C), 145.0 (C), 133.9 (CH), 132.3 (CH), 123.9 (d, ²J_{c,F}=18.4 Hz, CH), 109.4 (CH), 103.5 (C), 102.9 (CH), 81.7 (d, ³J_{c,F}=3.8 Hz, CH), 74.8 (CH), 74.2 (CH), 36.6 (CH₂), 33.4 (d, ³J_{c,F}=6.1 Hz, CH₂), 20.9 (CH₃); HRMS (MALDI-TOF) m/z [M+Na]⁺ calcd for C₁₈H₁₉FO₇Na: 389.1013. found: 389.1024.

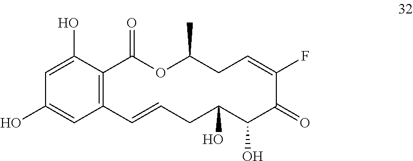 [SMILES: C[C@H]1C/C=C(/F)C(=O)[C@H](O)[C@H](O)C/C=C/c2cc(O)cc(O)c2C(=O)O1]

¹H-NMR (MeOD, 400 MHz): δ=6.63 (d, J=15.3 Hz, 1H), 6.49 (d, J=2.4 Hz, 1H), 6.30 (ddd, J=15.3, 8.6, 6.7 Hz, 1H), 6.26 (d, J=2.4 Hz, 1H), 6.19 (ddd, J=22.8, 11.0, 4.0 Hz, 1H), 5.39-5.28 (m, 1H), 4.90 (bs, 1H), 4.15-4.09 (m, 1H), 3.55-3.43 (m, 1H), 2.59 (dq, J=17.2, 4.4 Hz, 1H), 2.34-2.21 (m, 2H), 1.54 (d, J=6.2 Hz, 3H) ppm, 4OH signals not visible; ¹³C NMR (MeOD, 100 MHz, 25° C.): δ=196.7 (d, ²J_{c,F}=36.7 Hz, C═O), 171.9 (C═O), 164.4 (C), 163.4 (C), 155.4 (2×d, ¹J_{c,F}=253.8 Hz, C), 142.7 (C), 132.7 (CH), 130.3 (CH), 117.5 (d, ²J_{c,F}=19.1 Hz, CH), 108.0 (CH), 106.2 (C), 102.7 (CH), 80.8 (d, ³J_{c,F}=3.7 Hz, CH), 73.8 (d, ³J_{c,F}=2.2 Hz, CH), 72.7 (CH), 37.0 (CH₂), 31.8 (d, ³J_{c,F}=5.9 Hz, CH₂), 17.8 (CH₃); HRMS (MALDI-TOF) m/z [M+Na]⁺ calcd for C₁₈H₁₉FO₇Na: 389.1013. found: 389.1015.

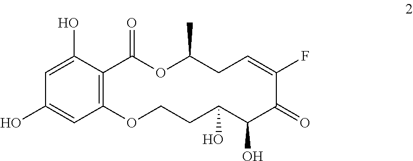 [SMILES: C[C@H]1C/C=C(/F)C(=O)[C@@H](O)[C@@H](O)CCOc2cc(O)cc(O)c2C(=O)O1]

Macrocycle 2: To a solution of cis enone 20 (1.0 equiv, 26 mg, 0.049 mmol) in MeOH (3 mL) was added PS—SO₃H resin (10.0 equiv, 164 mg, 0.493 mmol, 3.0 mmol/g) at 23° C. The reaction mixture was shaken for 7 hours, then it was filtered washed with CH₂Cl₂ (5×2 mL) and the solvents were removed under reduced pressure. The residue was purified by flash chromatography (CH₂Cl₂/MeOH, 25:1) to obtain desired macrocycle as a mixture of isomers (435 mg, 16 mg, 88%), which were separated by HPLC [Supelco C18 (5 cm×10 mm, 5 uM) 16 to 20% acetonirtrile in water, 2 mL/min] to obtain pure macrocycles 2 and 33 in 60 and 20% yield respectively.

2: R_{f}=0.18 (CH₂Cl₂/MeOH, 9:1); ¹H-NMR (MeOD, 400 MHz): δ=6.02 (d, J=1.9 Hz, 1H), 5.98 (d, J=1.9 Hz, 1H), 5.88 (ddd, J=21.8, 11.8, 2.7 Hz, 1H), 5.44-5.29 (m, 1H), 5.03 (d, J=4.3 Hz, 1H), 4.24-4.18 (m, 2H), 3.97-3.88 (m, 1H), 3.11-3.00 (m, 1H), 2.55-2.44 (m, 1H), 1.94-1.86 (m, 1H), 1.74-1.61 (m, 1H), 1.38 (d, J=5.9 Hz, 3H) ppm, 4OH signals not visible; ¹³C NMR (MeOD, 100 MHz, 25° C.): δ=196.8 (d, ²J_{c,F}=35.9 Hz, C═O), 172.6 (C═O), 167.1 (C), 165.4 (C), 163.6 (C), 155.6 (2×d, ¹J_{c,F}=255.3 Hz, C), 119.5 (d, ²J_{c,F}=17.6 Hz, CH), 96.6 (CH), 96.4 (C), 92.8 (CH), 80.8 (d, ³J_{c,F}=2.9 Hz, CH), 73.1 (CH), 69.5 (CH), 65.8 (CH₂), 33.1 (d, ³J_{c,F}=5.9 Hz, CH₂), 31.3 (CH₂), 20.8 (CH₃); HRMS (MALDI-TOF) m/z [M+Na]⁺ calcd for C₁₇H₁₉FO₈Na: 393.0962. found: 393.0942.

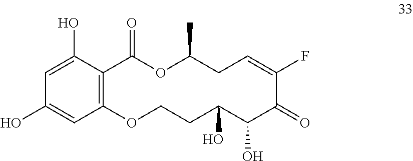 [SMILES: C[C@H]1C/C=C(/F)C(=O)[C@H](O)[C@H](O)CCOc2cc(O)cc(O)c2C(=O)O1]

¹H-NMR (MeOD, 400 MHzδ=6.21 (ddd, J=23.4, 11.3, 4.6 Hz, 1H), 6.04 (d, J=2.1 Hz, 1H), 5.99 (d, J=2.1 Hz, 1H), 5.34-5.25 (m, 1H), 5.05 (dd, J=4.3, 1.6 Hz, 1H), 4.36-4.25 (m, 2H), 3.89-3.82 (m, 1H), 3.21 (ddd, J=15.3, 11.1, 2.9 Hz, 1H), 2.54 (dq, J=16.4, 4.4 Hz, 1H), 1.89 (ddd, J=15.0, 10.4, 4.2 Hz, 1H), 1.68-1.62 (m, 1H), 1.32 (d, J=6.2 Hz, 3H) ppm, 4OH signals not visible; ¹³C NMR (MeOD, 100 MHz, 25° C.): δ=196.3 (d, ²J_{c,F}=35.3 Hz, C═O), 172.0 (C═O), 165.5 (C), 164.7 (C), 163.0 (C), 155.3 (2×d, ¹J_{c,F}=252.3 Hz, C), 117.1 (d, ²J_{c,F}=19.2 Hz, CH), 98.3 (CH), 96.8 (C), 93.7 (CH), 80.7 (d, ³J_{c,F}=2.3 Hz, CH), 72.2 (CH), 69.9 (CH), 66.9 (CH₂), 31.7 (d, ³J_{c,F}=6.1 Hz, CH₂), 31.6 (CH₂), 17.9 (CH₃); HRMS (MALDI-TOF) m/z [M+Na]⁺ calcd for C₁₇H₁₉FO₈Na: 393.0962. found: 393.0985.

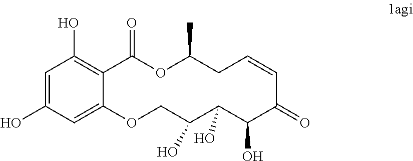 [SMILES: C[C@H]1C/C=C\C(=O)[C@@H](O)[C@@H](O)[C@H](O)COc2cc(O)cc(O)c2C(=O)O1]

1agi: Rf=0.6 (CH2Cl2/MeOH 4/1); 1H NMR (CD3OD, 400 MHz, 25° C.) d 6.45 (dd, J=11.8, 2.7 Hz, 1H), 6.15 (td, J=11.8, 2.7 Hz, 1H), 5.93 (d, J=2.54 Hz, 1H), 5.92 (d, J=2.4 Hz, 1H), 5.36-5.29 (m, 1H), 4.74 (d, J=1.3 Hz, 1H), 4.13-4.09 (m, 1H), 4.03 (d, J=1.4 Hz, 1H), 3.82 (dd, J=9.7, 2.7 Hz, 1H), 3.56 (dd, J=8.6, 2.7 Hz, 1H), 3.31 (dt, J=16.5, 11.3 Hz, 1H), 2.43 (dq, J=16.6, 2.4 Hz, 1H), 1.38 (d, J=5.9 Hz, 3H), 5OH signals are not visible. HRMS (MALDI-TOF) m/z [M+H]⁺ calcd for C17H20O9H, 369.1186. found: 369.1180.

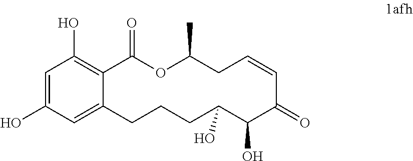 [SMILES: C[C@H]1C/C=C\C(=O)[C@@H](O)[C@@H](O)CCCc2cc(O)cc(O)c2C(=O)O1]

1afh: Rf=0.29 (CH2Cl2/MeOH 10/1)); 1H NMR (CD3OD, 400 MHz, 25° C.) d 6.47 (dd, J=11.6 Hz, 2.4 Hz, 1H), 6.24 (td, J=11.3 Hz, 2.4 Hz, 1H), 6.21 (d, J=2.1 Hz, 1H), 6.15 (d, J=2.4 Hz, 1H), 5.43-5.35 (m, 1H), 4.49 (d, J=1.6 Hz, 1H), 3.93-3.87 (m, 1H), 3.04-2.92 (m, 1H), 2.69-2.49 (m, 2H), 1.85-1.72 (m, 1H), 1.70-1.56 (m, 2H), 1.39 (d, J=6.4 Hz, 3H), 1.39-1.35 (m, 2H), 4OH signals are not visible. HRMS (MALDI-TOF) m/z [M+H]+ calcd for C18H22O7H, 351.1444. found: 351.1429.

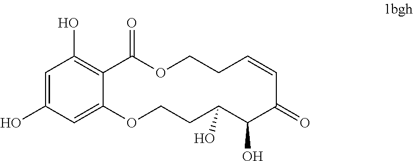 [SMILES: O=C1OCC/C=C\C(=O)[C@@H](O)[C@@H](O)CCOc2cc(O)cc(O)c21]

1bgh: Rf=0.51 (EtOAc/MeOH 10/1); 1H NMR (CD3OD, 400 MHz, 25° C.) d 6.48 (dd, J=11.6, 2.8 Hz, 1H), 6.15 (td, J=11.2, 2.4 Hz, 1H), 5.94 (d, J=2.0 Hz, 1H), 5.90 (d, J=2.0 Hz, 1H), 4.63 (s, 1H), 4.56 (t, J=11.6 Hz, 2H), 4.22-4.14 (m, 2H), 3.81-3.78 (m, 1H), 2.49-2.44 (m, 1H), 1.85-1.78 (m, 1H), 1.61-1.55 (m, 2H), 4OH signals are not visible. HRMS (MALDI-TOF) m/z [M+H]+ calcd for C16H18O8H, 339.1080. found: 339.1056

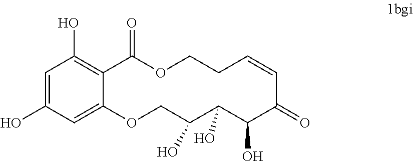 [SMILES: O=C1OCC/C=C\C(=O)[C@@H](O)[C@@H](O)[C@H](O)COc2cc(O)cc(O)c21]

1bgi: Rf=0.64 (CH2Cl2/MeOH 4/1); 1H NMR (CD3OD, 400 MHz, 25° C.) d 6.50 (dd, J=11.8, 2.7 Hz, 1H), 6.17 (td, J=11.3, 2.7 Hz, 1H), 5.95-5.92 (m, 2H), 4.77 (s, 1H), 4.62-4.52 (m, 2H), 4.20-4.12 (m, 2H), 3.88 (dd, J=9.7, 2.7 Hz, 1H), 3.54 (dd, J=8.0, 2.7 Hz, 1H), 2.54-2.45 (m, 1H), 1.99-1.85 (m, 1H), 5OH signals are not visible. HRMS (MALDI-TOF) m/z [M+H]+ calcd for C16H18O9H, 355.1029. found: 355.1054.

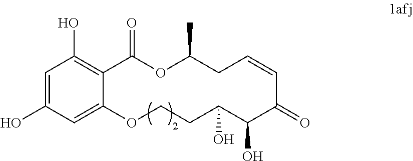

1afj: Rf=0.26 (CH2Cl2/MeOH 10/1); 1H NMR (CD3OD, 400 MHz, 25° C.) δ 6.46 (dd, J=11.6 Hz, 2.4 Hz, 1H), 6.29 (td, J=10.4 Hz, 2.4 Hz, 1H), 6.22 (d, J=2.4 Hz, 1H), 6.15 (d, J=2.4 Hz, 1H), 5.55-5.49 (m, 1H), 4.48 (d, J=1.6 Hz, 1H), 4.05-4.02 (m, 1H), 3.38-3.18 (m, 1H), 2.66-2.61 (m, 1H), 2.35-2.28 (m, 1H), 1.82-1.74 (m, 1H), 1.66-1.56 (m, 2H), 1.40 (d, J=6.0 Hz, 3H), 1.36-1.29 (m, 4H), 4OH signals are not visible. HRMS (MALDI-TOF) m/z [M+H]⁺ calcd for C₁₉H₂₄O₇H, 351.1444. found: 351.1469.

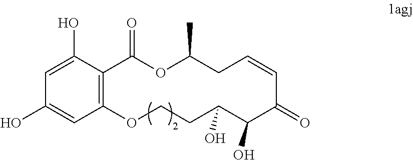

1agj: Rf=0.43 (15% MeOH/CH₂Cl₂); ¹H-NMR (CDCl₃, 400 MHz, 25° C.) δ 12.24 (s, 1H), 6.26 (dd, J=11.2, 2.9 Hz, 1H), 6.10 (td, J=11.3, 2.1 Hz, 1H), 6.03 (s, 1H), 5.93 (d, J=2.1 Hz, 1H), 5.49-5.43 (m, 1H), 4.57 (d, J=3.5 Hz, 1H), 4.05-3.92 (m, 3H), 2.99-2.89 (m, 1H), 2.63-2.57 (m, 1H), 1.92-1.83 (m, 2H), 1.59-1.43 (m, 2H), 1.38 (d, J=5.9 Hz, 3H), 3OH signals are not visible. HRMS (MALDI-TOF) m/z [M+H]⁺ calcd for C₁₈H₂₂O₈H, 367.1393; found: 367.1378.

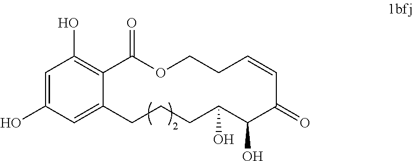

1bfj: Rf=0.24 (CH₂Cl₂/MeOH 9/1); ¹H NMR (CD₃OD, 400 MHz, 25° C.) δ 6.61 (d, J=12.0 Hz, 1H), 6.34-6.28 (m, 1H), 6.23 (d, J=2.0 Hz, 1H), 6.14 (d, J=2.4 Hz, 1H), 4.66-4.39 (m, 3H), 4.01-3.98 (m, 1H), 2.92-2.71 (m, 3H), 2.61-2.53 (m, 1H), 1.67-1.58 (m, 2H), 1.47-1.38 (m, 2H), 1.36-1.29 (m, 2H), 4OH signals are not visible. HRMS (MALDI-TOF) m/z [M+H]⁺ calcd for C₁₈H₂₂O₇H, 351.1444. found: 351.1469.

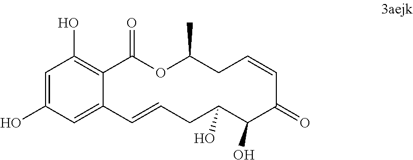 [SMILES: C[C@H]1C/C=C\C(=O)[C@@H](O)[C@@H](O)C/C=C/c2cc(O)cc(O)c2C(=O)O1]

3aejk. Rf=0.44 (3% MeOH in EtOAc); ¹H NMR (CDCl₃, 500 MHz, 25° C.) 12.15 (s, 1H), 6.87 (d, J=15.1 Hz, 1H), 6.40 (d, J=2.4 Hz, 1H), 6.38 (d, J=2.4 Hz, 1H), 6.33 (dd, J=11.4, 2.6 Hz, 1H), 6.20 (td, J=11.1, 2.6 Hz, 1H), 5.98 (ddd, J=15.1, 8.4, 4.1 Hz, 1H), 5.27-5.21 (m, 1H), 4.50 (bs, 1H), 4.00-3.98 (m, 1H), 3.81 (s, 3H), 3.58 (dt, J=17.0, 11.3 Hz, 1H), 2.50 (dq, J=17.0, 2.1 Hz, 1H), 2.23-2.19 (m, 1H), 2.13 (ddd, J=15.7, 10.8, 3.0 Hz, 1H), 1.47 (d, J=6.1 Hz, 3H), 2OH signals no visible; ¹³C NMR (CDCl₃, 125.75 MHz, 25° C.) 199.2, 171.5, 166.1, 164.4, 147.5, 143.1, 132.9, 130.2, 125.3, 108.2, 103.5, 100.4, 80.8, 73.8, 73.7, 55.6, 37.6, 37.2, 20.9; HRMS (MALDI-TOF) m/z 385.1260 ([M+Na]⁺, C₁₉H₂₂O₇Na requires 385.1264).

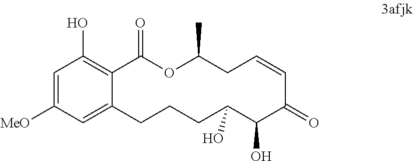 [SMILES: COc1cc(O)c2c(c1)CCC[C@H](O)[C@H](O)C(=O)/C=C\C[C@H](C)OC2=O]

3afjk. ¹H NMR (CDCl₃, 400 MHz, 25° C.) 12.18 (s, 1H), 6.39 (dd, J=11.6, 3.2 Hz, 1H), 6.36 (d, J=2.4 Hz, 1H), 6.29 (dd, J=11.6, 2.4 Hz, 1H), 6.28 (d, J=2.4 Hz, 1H), 5.48-5.40 (m, 1H), 4.56-4.54 (m, 1H), 4.21 (ddd, J=16.4, 12.0, 4.8 Hz, 1H), 3.93 (bs, 1H), 3.82 (s, 3H), 3.75 (d, J=6.8 Hz, 1H), 3.68-3.65 (m, 2H), 3.37 (dt, J=17.5, 11.5 Hz, 1H), 2.96 (tm, J=12.0 Hz, 1H), 2.57 (dq, J=16.8, 2.8 Hz, 1H), 2.52-2.44 (m, 1H), 2.04-2.00 (m, 1H), 1.45 (d, J=6.0 Hz, 3H), 1.12 (m, 1H); HRMS (MALDI-TOF) m/z 365.1637 ([M+H+], C₁₉H₂₄O₇H requires 365.1600).

1-18. (canceled)

19. A compound of formula II, a tautomer or pharmaceutically acceptable salt, solvate, or ester thereof, 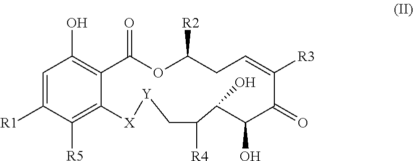
wherein:
R1 is selected from the group consisting of hydrogen, halogen, cyano, —OR, —NRR′, —NRS(O)R′, —NRS(O₂)R′, —SR, —S(O)R, —S(O₂)R, —OC(O)R, —C(O)R, —C(O)OR, —NRC(O)R′, —C(O)NRR′, —OC(O)OR, aliphatic, heteroaliphatic, acyl, aryl, heteroaryl, alkylaryl, arylalkyl, alkylheteroaryl and heteroarylalkyl;
R2 is hydrogen, or a C₁-C₅ alkyl;
R3 is hydrogen or halogen;
R4 is hydrogen or hydroxyl;
R5 is hydrogen, halogen, OR, NHR, NH—COR;
R and R′, independently, are hydrogen, or a C₁-C₅ alkyl; and
X—Y is CH═CH, CH₂—CH₂, O—CH₂, CH₂—O, S—CH₂, or CH₂—S.

20. The compound of claim 19, wherein R1 is selected from the group consisting of —OR, —NHR and —SR.

21. The compound of claim 19, wherein:
R1 is —OR and R is selected from hydrogen, methyl, ethyl and isopropyl; and/or
R2 is selected from hydrogen, methyl, ethyl and isopropyl; and/or
R3 is hydrogen or fluorine; and/or
R4 is hydrogen or hydroxyl; and/or
R5 is hydrogen; and/or
X—Y is selected from CH═CH, CH₂—CH₂, O—CH₂, CH₂—O, S—CH₂, and CH₂—S; provided that the compound does not have concomitantly a hydrogen in R2 and O—CH₂ in X—Y.

22. The compound of claim 19, wherein:
R1 is hydroxyl or methoxy; and/or
R2 is hydrogen or methyl; and/or
R3 is hydrogen; and/or
R4 is hydrogen; and/or
R5 is hydrogen; and/or
X—Y is selected from CH═CH, CH₂—CH₂, and O—CH₂;
provided that the compound does not have concomitantly a hydrogen in R2 and O—CH₂ in X—Y.

23. The compound of claim 19, wherein X—Y is CH₂—CH₂.

24. The compound of claim 19, wherein R1 is hydroxyl or methoxy, R2, R3, R4 and R5 are hydrogen, and X—Y is CH═CH or CH₂—CH₂.

25. The compound of claim 19, wherein R1 is hydroxyl or methoxy, R2 is methyl, R3, R4 and R5 are hydrogen, and X—Y is selected from CH═CH, CH₂—CH₂, and O—CH₂.

26. The compound of claim 19, wherein the compound is selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and
a compound of formula II, wherein R2, R3, R4 and R5 are hydrogen, R1 is methoxy and X—Y is CH₂—CH₂.

27. A pharmaceutical composition comprising a compound according to claim 19 and a pharmaceutically acceptable carrier.

28. The pharmaceutical composition of claim 27, wherein said compound is selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and
a compound of formula II, wherein R2, R3, R4 and R5 are hydrogen, R1 is methoxy and X—Y is CH₂—CH₂.

29. A method for treating a cancer in a subject wherein the method comprises administering a therapeutically effective amount of a compound according to claim 19 to a subject having cancer.

30. The method of claim 29, wherein said compound is selected from the group consisting of:
a compound of formula II, wherein R1 is hydroxyl, R3, R4 and R5 are hydrogen, R2 is methyl, and X—Y is CH═CH;
a compound of formula II, wherein R1 is hydroxyl, R3, R4 and R5 are hydrogen, R2 is methyl, and X—Y is CH₂—CH₂;
a compound of formula II, wherein R1 is hydroxyl, R3, R4 and R5 are hydrogen, R2 is methyl, and X—Y is O—CH₂;
a compound of formula II, wherein R1 is hydroxyl, R2, R3, R4 and R5 are hydrogen, and X—Y is CH₂—CH₂;
a compound of formula II, wherein R3, R4 and R5 are hydrogen, R1 is methoxy, R2 is methyl, and X—Y is CH═CH;
a compound of formula II, wherein R3, R4 and R5 are hydrogen, R1 is methoxy, R2 is methyl, and X—Y is CH₂—CH₂; and
a compound of formula II, wherein R2, R3, R4 and R5 are hydrogen, R1 is methoxy and X—Y is CH₂—CH₂.

31. The method of claim 29, wherein the method allows the treatment of cancer and reduces metastasis.
